(19) United States
(12) Patent Application Publication
(10) Pub. No.: US 2023/0059845 A1
(43) Pub. Date: Feb. 23, 2023

(54) PHOTOTHERAPEUTIC TREATMENT OF SKIN DISORDERS

(71) Applicant: KNOW Bio, LLC, Durham, NC (US)

(72) Inventors: Nathan Stasko, Chapel Hill, NC (US); Nicholas William Medendorp, Jr., Raleigh, NC (US); Thomas Matthew Womble, Wake Forest, NC (US)

(21) Appl. No.: 17/758,662

(22) PCT Filed: Jan. 16, 2021
(86) PCT No.: PCT/US21/13773

§ 371 Date: Jul. 12, 2022

Related U.S. Application Data
(60) Provisional application No. 62/962,642, filed on Jan. 17, 2020.

(51) Int. Cl. A61N 5/06

(52) U.S. Cl. CPC A61N 5/0616; A61N 2005/0629; A61N 2005/0652; A61N 2005/0661; A61N 2005/0663; A61N 2005/0647; A61N 2005/0665

(57) ABSTRACT

Methods of treating skin disorders are disclosed. The methods involve impinging light having a first peak wavelength on the tissue at a first radiant flux, wherein the first peak wavelength and the first radiant flux is selected to provide an anti-inflammatory effect, and impinging light having a second peak wavelength on the tissue at a second radiant flux, wherein the second peak wavelength and the second radiant flux are selected to either stimulate enzymatic generation of nitric oxide to increase endogenous stores of nitric oxide or release nitric oxide from the endogenous stores are disclosed. Representative skin disorders include pruritus, psoriasis, acne, rosacea, and eczema, and the skin can include the scalp. The methods can reduce stinging and/or itching associated with the skin disorder. The anti-inflammatory wavelengths can be in the range of between about 650 and about 680 nm.

### CROSS-REFERENCE TO RELATED APPLICATIONS

The present patent application claims the benefit and priority of U.S. Provisional Patent Application No. 62/962,642 filed on Jan. 17, 2020, titled “PHOTOTHERAPEUTIC TREATMENT OF SKIN DISORDERS,” the contents of which is hereby incorporated by reference in its entirety.

### TECHNICAL FIELD

This disclosure relates to systems and methods for treating skin disorders, such as pruritis and psoriasis, by using a combination of wavelengths of light to a) stimulate nitric oxide production and/or release in skin tissues of mammalian subjects, and b) provide an anti-inflammatory effect on the skin tissues.

### BACKGROUND

The term “phototherapy” relates to the therapeutic use of light. Various light therapies (e.g., including low level light therapy (LLLT) and photodynamic therapy (PDT)) have been publicly reported or claimed to provide various health related medical benefits. These benefits include promoting hair growth; treating skin or tissue inflammation; promoting tissue or skin healing or rejuvenation; enhancing wound healing; wrinkle reduction, scar reduction, as well as a treating stretch marks, varicose veins, and spider veins.

Various mechanisms by which phototherapy has been suggested to provide therapeutic benefits include: increasing circulation (e.g., by increasing formation of new capillaries); stimulating the production of collagen; stimulating the release of adenosine triphosphate (ATP); enhancing porphyrin production; reducing excitability of nervous system tissues; stimulating fibroblast activity; increasing phagocytosis; inducing thermal effects; stimulating tissue granulation and connective tissue projections; reducing inflammation; and stimulating acetylcholine release.

Phototherapy has also been suggested to stimulate cells to generate nitric oxide. Various biological functions attributed to nitric oxide include roles as signaling messenger, cytotoxin, antiapoptotic agent, antioxidant, and regulator of microcirculation Nitric oxide is recognized to relax vascular smooth muscles, dilate blood vessels, inhibit aggregation of platelets, and modulate T cell-mediate immune response.

Nitric oxide is produced by multiple cell types in skin, and is formed by the conversion of the amino acid L-arginine to L-citrulline and nitric oxide, mediated by the enzymatic action of nitric oxide synthases (NOSs). NOS is a NADPH-dependent enzyme that catalyzes the following reaction:

L-arginine+3/2 NADPH+H⁺+2 O₂ [symbol] citrulline+nitric oxide+3/2 NADP⁺

In mammals, three distinct genes encode NOS isozymes: neuronal (nNOS or NOS-I), cytokine-inducible (iNOS or NOS-II), and endothelial (eNOS or NOS-III). iNOS and nNOS are soluble and found predominantly in the cytosol, while eNOS is membrane associated. Many cells in mammals synthesize iNOS in response to inflammatory conditions.

Skin has been documented to upregulate inducible nitric oxide synthase expression and subsequent production of nitric oxide in response to irradiation stress. Nitric oxide serves a predominantly anti-oxidant role in the high levels generated in response to radiation.

Nitric oxide is a free radical capable of diffusing across membranes and into various tissues; however, it is very reactive, with a half-life of only a few seconds. Due to its unstable nature, nitric oxide rapidly reacts with other molecules to form more stable products. For example, in the blood, nitric oxide rapidly oxidizes to nitrite, and is then further oxidized with oxyhaemoglobin to produce nitrate. Nitric oxide also reacts directly with oxyhaemoglobin to produce methaemoglobin and nitrate. Nitric oxide is also endogenously stored on a variety of nitrosated biochemical structures including nitrosoglutathione (GSNO), nitrosoalbumin, nitrosohemoglobin, and a large number of nitrosocysteine residues on other critical blood/tissue proteins. The term “nitroso” is defined as a nitrosated compound (RSNO or RNNO), via either S- or N-nitrosation. Metal nitrosyl (M-NO) complexes are another endogenous store of circulating nitric oxide, most commonly found as ferrous nitrosyl complexes in the body; however, metal nitrosyl complexes are not restricted to complexes with iron-containing metal centers. Nitric oxide loaded chromophores including cytochrome c oxidase (CCO—NO) represent additional endogenous stores of nitric oxide.

When nitric oxide is auto-oxidized into nitrosative intermediates, the nitric oxide is bound covalently in the body (in a “bound” state). Thus, conventional efforts to produce nitric oxide in tissue may have a limited therapeutic effect, since nitric oxide in its “gaseous” state is short-lived, and cells being stimulated to produce nitric oxide may become depleted of NADPH or L-Arginine to sustain nitric oxide production.

While light therapy associated with nitric oxide release may be useful in treating certain disorders, it would be advantageous to have additional therapeutic methods.

### SUMMARY

In one embodiment, methods of treating skin disorders, comprising treating the skin with two different wavelengths, is disclosed. Light having a first peak wavelength and a first radiant flux stimulates enzymatic generation of nitric oxide to increase endogenous stores of nitric oxide, or releases nitric oxide from the endogenous stores. Light having a second peak wavelength and a second radiant flux provides an anti-inflammatory effect.

Representative skin disorders include pruritus, psoriasis, acne, rosacea, eczema, such as eczema verruca vulgaris, neurofibromatosis, pyogenic granulomas, recessive dystrophic epidermolysis bullosa, venous ulcers, molluscum contagiosum, seborrheic keratosis, Sturge-Weber syndrome, actinic keratosis, and dandruff. In one embodiment, the skin disorder is pruritus, psoriasis, acne, rosacea, or eczema. In another embodiment, the skin disorder is a disorder of the skin of the scalp, such as pruritis or psoriasis.

The method includes impinging light having the first peak wavelength on the tissue at a first radiant flux, and impinging light having the second peak wavelength on the tissue at a second radiant flux. In one aspect of this method, the first and second wavelength are impinged simultaneously, and in another aspect of this method, the first and second wavelength are impinged in alternation.

In certain embodiments, the second peak wavelength is greater than the first peak wavelength by at least 25 nm.

In certain embodiments, each of the first radiant flux and the second radiant flux is in a range of from 5 mW/cm² to 60 mW/cm².

In another aspect, the disclosure relates to a device for treating skin disorders. The device includes means for impinging light having the first peak wavelength on the tissue at a first radiant flux, and for impinging light having the second peak wavelength on the tissue at a second radiant flux.

In certain embodiments, the device further includes driver circuitry configured to drive the at least one first light emitting device and the at least one second light emitting device.

In some embodiments, the device includes at least one first solid state light emitting device configured to impinge light having the first peak wavelength on tissue, and can further comprise at least one second solid state light emitting device configured to impinge light having the second peak wavelength on the tissue. The device additionally includes driver circuitry configured to drive the at least one first solid state light emitting device and the at least one second solid state light emitting device.

In certain embodiments of the device, each of the first radiant flux and the second radiant flux is in a range of from 5 mW/cm² to 60 mW/cm².

The first peak wavelength is selected to provide nitric oxide production or release. In some embodiments, a third wavelength is used, so as to provide both nitric oxide production and release.

The second peak wavelength is between about 650 nm and about 680 nm, more specifically, between about 655 nm and about 675 nm, still more specifically, around 660 nm.

In one embodiment, the first peak wavelength is in a range of from 615 nm to 640 nm and the second peak wavelength is in a range of from 650 nm to 670 nm. In one aspect of this embodiment, the first peak wavelength is in a range of from 620 nm to 625 nm and the second peak wavelength is in a range of from 655 nm to 665 nm.

In another aspect, any of the foregoing aspects, and/or various separate aspects and features as described herein, may be combined for additional advantage. Any of the various features and elements as disclosed herein may be combined with one or more other disclosed features and elements unless indicated to the contrary herein.

Other aspects, features and embodiments of the invention will be more fully apparent from the ensuing disclosure and the appended claims.

### BRIEF DESCRIPTION OF DRAWINGS

FIG. 1 is a side cross-sectional schematic view of a portion of a device for delivering light energy to living mammalian tissue, the device including multiple direct view light emitting sources supported by a substrate and covered with an encapsulant material layer.

FIG. 2 is a side cross-sectional schematic view of a portion of a device for delivering light energy to living mammalian tissue, the device including multiple direct view light emitting sources supported by a substrate and covered with an encapsulant material layer, wherein at least one functional material (e.g., wavelength conversion and/or scattering material) is disposed within the encapsulant material layer.

FIG. 3 is a side cross-sectional schematic view of a portion of a device for delivering light energy to living mammalian tissue, the device including multiple direct view light emitting sources supported by a substrate and covered with two encapsulant material layers, with at least one functional material (e.g., wavelength conversion and/or scattering material) layer disposed between the encapsulant material layers.

FIG. 4 is a side cross-sectional schematic view of a portion of a device for delivering light energy to living mammalian tissue, the device including multiple direct view light emitting sources supported by a substrate and covered by an encapsulant layer, wherein the encapsulant layer is covered with a diffusion or scattering material layer.

FIG. 5 is a side cross-sectional schematic view of a portion of a device for delivering light energy to living mammalian tissue, the device including multiple direct view light emitting sources supported by a substrate, multiple molded features overlying the light emitting sources, and an encapsulant or light coupling material arranged between the light emitting sources and the molded features.

FIG. 6 is a side cross-sectional schematic view of a portion of a device for delivering light energy to living mammalian tissue, the device including a flexible substrate, one or more organic light emitting diode layers arranged between an anode and cathode, and an encapsulant layer arranged over the cathode.

FIG. 7 is a side cross-sectional schematic view of a portion of a device for delivering light energy to living mammalian tissue, the device including a flexible substrate, multiple direct view light emitting sources supported by the substrate, encapsulant material layers arranged above and below the substrate and over the light emitting sources, and holes or perforations defined through both the substrate and the encapsulant material layers.

FIG. 8 is a side cross-sectional schematic view of a portion of a device for delivering light energy to living mammalian tissue, wherein the device includes multiple direct view light emitting sources supported by a substrate and covered by an encapsulant layer, and the device is arranged in a concave configuration.

FIG. 9 is a side cross-sectional schematic view of a portion of a device for delivering light energy to living mammalian tissue, wherein the device includes multiple direct view light emitting sources supported by a substrate and covered by an encapsulant layer, and the device is arranged in a convex configuration.

FIG. 10 is a side cross-sectional schematic view of a portion of a device for delivering light energy to living mammalian tissue, wherein the device is edge lit with one or more light emitting sources supported by a flexible printed circuit board (PCB), other non-light-transmitting surfaces of the device are bounded by a flexible reflective substrate, and the flexible PCB and light emitting source(s) are covered with an encapsulant material.

FIG. 11 is a side cross-sectional schematic view of a portion of a device for delivering light energy to living mammalian tissue, wherein the device is edge lit with one or more light emitting sources supported by a flexible printed circuit board (PCB), an other non-light-transmitting surface of the device is bounded by a flexible reflective substrate, the flexible PCB and light emitting source(s) are covered with an encapsulant material, and the device is tapered in thickness.

FIG. 12 is a side cross-sectional schematic view of a portion of a device for delivering light energy to living mammalian tissue, wherein the device is edge lit with one or more light emitting sources supported by a flexible PCB having a reflective surface, non-light-transmitting surfaces of the device are further bounded by the flexible PCB, and the flexible PCB and light emitting source(s) are covered with an encapsulant material.

FIG. 13 is a side cross-sectional schematic view of a portion of a device for delivering light energy to living mammalian tissue, wherein the device is edge lit with one or more light emitting sources supported by a flexible PCB having a reflective surface, another non-light-transmitting surface of the device is further bounded by the flexible PCB, the flexible PCB and light emitting source(s) are covered with an encapsulant material, and the device is tapered in thickness.

FIG. 14 is a side cross-sectional schematic view of a portion of a device for delivering light energy to living mammalian tissue, wherein the device is edge lit with one or more light emitting sources supported by a flexible PCB having a reflective surface, other non-light-transmitting surfaces of the device are further bounded by the flexible PCB, the flexible PCB and light emitting source(s) are covered with an encapsulant material, and a light-transmitting face of the device includes a diffusing and/or scattering layer.

FIG. 15 is a side cross-sectional schematic view of a portion of a device for delivering light energy to living mammalian tissue, wherein the device is edge lit with one or more light emitting sources supported by a flexible PCB having a reflective surface, another non-light-transmitting surface of the device is further bounded by the flexible PCB, the flexible PCB and light emitting source(s) are covered with an encapsulant material, a light transmitting face of the device is tapered in thickness, and the light-transmitting face includes a diffusing and/or scattering layer.

FIG. 16 is a side cross-sectional schematic view of a portion of a device for delivering light energy to living mammalian tissue, wherein the device is edge lit with one or more light emitting sources supported by a flexible PCB having a reflective surface, other non-light-transmitting surfaces of the device are further bounded by the flexible PCB, the flexible PCB and light emitting source(s) are covered with an encapsulant material, and a light-transmitting face of the device includes a wavelength conversion material layer.

FIG. 17 is a side cross-sectional schematic view of a portion of a device for delivering light energy to living mammalian tissue, wherein the device is edge lit with one or more light emitting sources supported by a flexible PCB having a reflective surface, another non-light-transmitting surface of the device is further bounded by the flexible PCB, the flexible PCB and light emitting source(s) are covered with an encapsulant material, a light transmitting face of the device is tapered in thickness, and the light-transmitting face includes a wavelength conversion material layer.

FIG. 18 is a side cross-sectional schematic view of a portion of a device for delivering light energy to living mammalian tissue, wherein the device is edge lit along multiple edges with multiple light emitting sources supported by a flexible PCB having a reflective surface, other non-light-transmitting surfaces of the device are further bounded by the flexible PCB, the flexible PCB and light emitting sources are covered with an encapsulant material, and a wavelength conversion material is distributed in the encapsulant material.

FIG. 19 is a side cross-sectional schematic view of a portion of a device for delivering light energy to living mammalian tissue, wherein the device is edge lit along multiple edges with multiple light emitting sources supported by a flexible PCB having a reflective surface, other non-light-transmitting surfaces of the device are further bounded by the flexible PCB with raised light extraction features being supported by the flexible PCB, and encapsulant material is provided over the flexible PCB, the light emitting sources, and the light extraction features.

FIG. 20 is a side cross-sectional schematic view of a portion of a device for delivering light energy to living mammalian tissue, wherein the device is edge lit along multiple edges with multiple light emitting sources supported by a flexible PCB having a reflective surface, other non-light-transmitting surfaces of the device are further bounded by the flexible PCB, an encapsulant material arranged above and below the PCB and over the light emitting sources, and holes or perforations are defined through both the substrate and the encapsulant material.

FIG. 21A is a cross-sectional view of a first exemplary hole definable through a device for delivering light energy to living mammalian tissue, the hole having a diameter that is substantially constant with depth.

FIG. 21B is a cross-sectional view of a second exemplary hole definable through a device for delivering light energy to living mammalian tissue, the hole having a diameter that increases with increasing depth.

FIG. 21C is a cross-sectional view of a second exemplary hole definable through a device for delivering light energy to living mammalian tissue, the hole having a diameter that decreases with increasing depth.

FIG. 22 is a top schematic view of at least a portion of a device for delivering light energy to living mammalian tissue, wherein the device is edge lit along multiple edges with multiple light emitting sources supported by a flexible PCB, and multiple holes or perforations of substantially uniform size and substantially uniform distribution are defined through the flexible PCB.

FIG. 23 is a top schematic view of at least a portion of a device for delivering light energy to living mammalian tissue, wherein the device is edge lit along multiple edges with multiple light emitting sources supported by a flexible PCB, and multiple holes or perforations of different sizes but with a substantially uniform distribution are defined through the flexible PCB.

FIG. 24 is a top schematic view of at least a portion of a device for delivering light energy to living mammalian tissue, wherein the device is edge lit along multiple edges with multiple light emitting sources supported by a flexible PCB, and multiple holes or perforations of different sizes are provided in clusters and defined through and the flexible PCB proximate to selected light emitting sources.

FIG. 25 is a top schematic view of at least a portion of a device for delivering light energy to living mammalian tissue, wherein the device is edge lit along multiple edges with multiple light emitting sources supported by a flexible PCB, and multiple holes or perforations of different sizes and with a non-uniform (e.g., random) distribution are defined through the flexible PCB.

FIG. 26A is a top schematic view of at least a portion of a light emitting device for delivering light energy to living mammalian tissue and at least a portion of a battery/control module, wherein an elongated electrical cord is associated with the battery/control module for connecting the battery/control module to the light emitting device.

FIG. 26B is a top schematic view of at least a portion of a light emitting device for delivering light energy to living mammalian tissue and at least a portion of a battery/control module, wherein an elongated electrical cord is associated with the light emitting device for connecting the light emitting device to the battery/control module.

FIG. 27 is a top schematic view of at least a portion of a light emitting device for delivering light energy to living mammalian tissue and being connected via an electrical cord to a battery/control module, wherein the light emitting device includes multiple light emitters, multiple holes or perforations, and multiple sensors.

FIG. 28A is a plot of intensity versus time embodying a first exemplary illumination cycle that may be used with at least one emitter of a light emitting device for delivering light energy to living mammalian tissue as disclosed herein.

FIG. 28B is a plot of intensity versus time embodying a second exemplary illumination cycle that may be used with at least one emitter of a light emitting device for delivering light energy to living mammalian tissue as disclosed herein.

FIG. 28C is a plot of intensity versus time embodying a third exemplary illumination cycle that may be used with at least one emitter of a light emitting device for delivering light energy to living mammalian tissue as disclosed herein.

FIG. 29 is an exploded view of a light emitting device embodied in a wearable cap for delivering light energy to a scalp of a patient, the device including at least one light emitter supported by a flexible PCB arranged in a concave configuration, a concave support member shaped to receive the flexible PCB and support a battery and control module, and a fabric covering arranged to cover the support member and flexible substrate.

FIG. 30 is a front elevation view of the light emitting device of FIG. 29 affixed to a modeled human head.

FIG. 31 is a bottom plan view of the flexible PCB of FIG. 29 prior to being shaped into a concave configuration.

FIG. 32 is a schematic diagram showing interconnections between components of a light emitting device or delivering light energy to tissue of a patient according to one embodiment.

FIG. 33 is a schematic diagram depicting an interface between hardware drivers, functional components, and a software application suitable for operating a light emitting device according to FIG. 32.

FIG. 34 is a schematic elevation view of at least a portion of a light emitting device for delivering light energy to tissue in an internal cavity of a patient according to one embodiment.

FIG. 35A is a schematic elevation view of at least a portion of a light emitting device including a concave light emitting surface for delivering light energy to cervical tissue of a patient according to one embodiment.

FIG. 35B illustrates the device of FIG. 43A inserted into a vaginal cavity to deliver light energy to cervical tissue of a patient.

FIG. 36A is a schematic elevation view of at least a portion of a light emitting device including a probe-defining light emitting surface for delivering light energy to cervical tissue of a patient according to another embodiment.

FIG. 36B illustrates the device of FIG. 36A inserted into a vaginal cavity, with a probe portion of the light-emitting surface inserted into a cervical opening, to deliver light energy to cervical tissue of a patient.

FIGS. 37A-C are charts showing the percentage of patients experiencing itching (37A), burning and/or stinging (37B) or irritation (37C), shown in terms of percentage (%) subjects versus a device providing light at 620 and 660 nm, and a “sham” device.

### DETAILED DESCRIPTION

Aspects of the disclosure relate to the treatment of skin diseases using light at two wavelengths. Light having a first peak wavelength and a first radiant flux either stimulates enzymatic generation of nitric oxide to increase endogenous stores of nitric oxide, or releases nitric oxide from the endogenous stores. Light having a second peak wavelength and a second radiant flux has an anti-inflammatory effect. The second peak wavelength differs from the first peak wavelength, and in one aspect, the second peak wavelength is at least 25 nm greater than the first peak wavelength.

Providing Anti-Inflammatory Effects and NO Stimulation and/or Release

The photoinitiated release of endogenous stores of nitric oxide (“NO”) effectively regenerates “gaseous” (or unbound) nitric oxide that was autooxidized into nitrosative intermediates and were bound covalently in the body in an “bound” state. By stimulating release of nitric oxide from endogenous stores, nitric oxide may be maintained in a gaseous state for an extended duration and/or a spatial zone of nitric oxide release may be expanded.

As noted previously, nitric oxide is endogenously stored on a variety of nitrosated biochemical structures. Upon receiving the required excitation energy, both nitroso and nitrosyl compounds undergo hemolytic cleavage of S—N, N—N, or M-N bonds to yield free radical nitric oxide. Nitrosothiols and nitrosamines are photoactive and can be phototriggered to release nitric oxide by wavelength specific excitation.

The effect of light at certain wavelengths in the production and/or release of nitric oxide is described in U.S. Pat. No. 10,525,275, the contents of which are hereby incorporated by reference.

It has been reported that NO may diffuse in mammalian tissue by a distance of up to about 500 microns. In certain embodiments, photons of a first energy hν₁ may be supplied to the tissue to stimulate enzymatic generation of NO to increase endogenous stores of NO in a first diffusion zone 1. Photons of a second energy hν₂ may be supplied to the tissue in a region within or overlapping the first diffusion zone 1 to trigger release of NO from endogenous stores, thereby creating a second diffusion zone 2. Alternatively, or additionally, photons of a second energy hν₂ may be supplied to stimulate enzymatic generation of NO to increase endogenous stores of NO in the second diffusion zone 2. Photons of a third energy hν₃ may be supplied to the tissue in a region within or overlapping the second diffusion zone 2 to trigger release of endogenous stores, thereby creating a third diffusion zone 3. Alternatively, or additionally, photons of a third energy hν₃ may be supplied to stimulate enzymatic generation of NO to increase endogenous stores of NO in the third diffusion zone 3. In certain embodiments, the first, second, and third diffusion zones 1-3 may have different average depths relative to an outer epidermal surface. In certain embodiments, the first photon energy hν₁, the second photon energy hν₂, and the third photon energy hν₃ may be supplied at different peak wavelengths, wherein different peak wavelengths may penetrate mammalian skin to different depths—since longer wavelengths typically provide greater penetration depth. In certain embodiments, sequential or simultaneous impingement of increasing wavelengths of light may serve to “push” a nitric oxide diffusion zone deeper within mammalian tissue than might otherwise be obtained by using a single (e.g., long) wavelength of light.

Light having a first peak wavelength and a first radiant flux that stimulates enzymatic generation of nitric oxide to increase endogenous stores of nitric oxide may be referred to herein as “endogenous store increasing light” or “ES increasing light.” Light having a first peak wavelength and a first radiant flux to release nitric oxide from the endogenous stores may be referred to herein as “endogenous store releasing light” or “ES releasing light.”

In certain embodiments, light at three peak wavelengths is used, including one peak wavelength to provide an anti-inflammatory effect, in combination with a peak wavelength of ES releasing light, and a peak wavelength of ES increasing light.

In certain embodiments, each of the anti-inflammatory light and ES increasing light and/or ES releasing light has a radiant flux in a range of at least 5 mW/cm², or at least 10 mW/cm², or at least 15 mW/cm², or at least 20 mW/cm², or at least 30 mW/cm², or at least 40 mW/cm², or at least 50 mW/cm², or in a range of from 5 mW/cm² to 60 mW/cm², or in a range of from 5 mW/cm² to 30 mW/cm², or in a range of from 5 mW/cm² to 20 mW/cm², or in a range of from 5 mW/cm² to 10 mW/cm², or in a range of from 10 mW/cm² to 60 mW/cm², or in a range of from 20 mW/cm² to 60 mW/cm², or in a range of from 30 mW/cm² to 60 mW/cm², or in a range of from 40 mW/cm² to 60 mW/cm², or in another range specified herein.

In certain embodiments, the ES increasing light has a greater radiant flux than the ES releasing light. In certain embodiments, the ES releasing light has a greater radiant flux than the ES increasing light. In certain embodiments, the anti-inflammatory light has a greater radiant flux than the ES increasing and/or ES releasing light. In certain other embodiments, the anti-inflammatory light has a lesser radiant flux than the ES increasing and/or ES releasing light.

In certain embodiments, one or both of the anti-inflammatory light and ES increasing and/or ES releasing light has a radiant flux profile that is substantially constant during a treatment window. In certain embodiments, at least one of the anti-inflammatory light and ES increasing and/or ES releasing light has a radiant flux profile that increases with time during a treatment window. In certain embodiments, at least one of the anti-inflammatory light and ES increasing and/or ES releasing light has a radiant flux profile that decreases with time during a treatment window. In certain embodiments, one of the anti-inflammatory light and ES increasing and/or ES releasing light has a radiant flux profile that decreases with time during a treatment window, while the other of the anti-inflammatory light and ES increasing and/or ES releasing light has a radiant flux profile that increases with time during a treatment window.

In certain embodiments, ES increasing and/or ES releasing light is applied to tissue during a first time window, and anti-inflammatory light is applied to the tissue during a second time window, and the second time window overlaps with the first time window. In other embodiments, ES increasing and/or ES releasing light is applied to tissue during a first time window, anti-flammatory light is applied to the tissue during a second time window, and the second time is non-overlapping with the first time window. In certain embodiments, the second time window is initiated more than one minute, more than 5 minutes, more than 10 minutes, more than 30 minutes, or more than one hour after conclusion of the first time window. In certain embodiments, ES increasing and/or releasing light is applied to tissue during a first time window, anti-inflammatory light is applied to the tissue during a second time window, and the first time window and the second time window are substantially the same. In other embodiments, the second time window is longer than the first time window.

In certain embodiments, one or both of the anti-inflammatory light and ES increasing light and/or ES releasing light may be provided by a steady state source providing a radiant flux that is substantially constant over a prolonged period without being pulsed.

In certain embodiments, one or both of anti-inflammatory light and ES increasing light and/or ES releasing light may include more than one discrete pulse of light. In certain embodiments, more than one discrete pulse of ES increasing and/or ES releasing light is impinged on tissue during a first time window, and/or more than one discrete pulse of anti-inflammatory light is impinged on tissue during a second time window. In certain embodiments, the first time window and the second time window may be coextensive, may be overlapping but not coextensive, or may be non-overlapping.

In certain embodiments, at least one of radiant flux and pulse duration of ES increasing and/or ES releasing light may be reduced from a maximum value to a non-zero reduced value during a portion of a first time window. In certain embodiments, at least one of radiant flux and pulse duration of ES increasing and/or ES releasing light may be increased from a non-zero value to a higher value during a portion of a first time window. In certain embodiments, at least one of radiant flux and pulse duration of anti-inflammatory light may be reduced from a maximum value to a non-zero reduced value during a portion of a second time window. In certain embodiments, at least one of radiant flux and pulse duration of anti-inflammatory light may be increased from a non-zero value to a higher value during a portion of a second time window.

In certain embodiments, each of ES increasing and/or releasing light and the anti-inflammatory light consist of non-coherent light. In certain embodiments, each of the anti-inflammatory light and the ES increasing light and/or ES releasing light consist of coherent light. In certain embodiments, one of the anti-inflammatory light and the ES increasing light and/or the ES releasing light consists of non-coherent light, and the other consists of coherent light.

In certain embodiments, the ES increasing and/or ES releasing light is provided by at least one first light emitting device and the anti-inflammatory light is provided by at least one second light emitting device. In certain embodiments, the ES increasing and/or ES releasing light is provided by a first array of light emitting devices and the anti-inflammatory light is provided by a second array of light emitting devices.

In certain embodiments, at least one of the ES increasing and/or ES releasing light and the anti-inflammatory light is provided by at least one solid state light emitting device. Examples of solid state light emitting devices include (but are not limited to) light emitting diodes, lasers, thin film electroluminescent devices, powdered electroluminescent devices, field induced polymer electroluminescent devices, and polymer light-emitting electrochemical cells. In certain embodiments, the ES increasing and/or ES releasing light is provided by at least one first solid state light emitting device and the anti-inflammatory light is provided by at least one second solid state light emitting device. In certain embodiments, the anti-inflammatory and the ES increasing light and/or ES releasing light may be generated by different emitters contained in a single solid state emitter package, wherein close spacing between adjacent emitters may provide integral color mixing. In certain embodiments, the anti-inflammatory light may be provided by a first array of solid state light emitting devices and the ES increasing and/or ES releasing light may be provided by a second array of solid state light emitting devices. In certain embodiments, an array of solid state emitter packages each including at least one first emitter and at least one second emitter may be provided, wherein the array of solid state emitter packages embodies a first array of solid state emitters arranged to generate ES increasing and/or ES releasing light and embodies a second array of solid state emitters arranged to generate anti-inflammatory light. In certain embodiments, an array of solid state emitter packages may embody packages further including third, fourth, and/or fifth solid state emitters, such that a single array of solid state emitter packages may embody three, four, or five arrays of solid state emitters, wherein each array is arranged to generate a emissions with a different peak wavelength.

In certain embodiments, at least one of anti-inflammatory and the ES increasing light and/or the ES releasing light is provided by at least one light emitting device devoid of a wavelength conversion material. In other embodiments, at least one of the anti-inflammatory and the ES increasing light and/or the ES releasing light is provided by at least one light emitting device arranged to stimulate a wavelength conversion material, such as a phosphor material, a fluorescent dye material, a quantum dot material, and a fluorophore material.

In certain embodiments, the anti-inflammatory light consists of substantially monochromatic light and the ES increasing light and/or ES releasing light consists of substantially monochromatic light. In certain embodiments, the ES increasing light includes a first spectral output having a first full width at half maximum value of less than 25 nm (or less than 20 nm, or less than 15 nm, or in a range of from 5 nm to 25 nm, or in a range of from 10 nm to 25 nm, or in a range of from 15 nm to 25 nm), and/or the ES releasing light includes a second spectral output having a second full width at half maximum value of less than 25 nm (or less than 20 nm, or less than 15 nm, or in a range of from 5 nm to 25 nm, or in a range of from 10 nm to 25 nm, or in a range of from 15 nm to 25 nm).

In certain embodiments, the anti-inflammatory light is produced by one or more first light emitters having a single first peak wavelength, and the ES increasing light and/or ES releasing light is produced by one or more second light emitters having a single second peak wavelength. In other embodiments, the anti-inflammatory light may be produced by at least two light emitters having different peak wavelengths (e.g., differing by at least 5 nm, at least 10 nm, at least 15 nm, at least 20 nm, or at least 25 nm), and/or the ES increasing and/or the ES releasing light may be produced by at least two light emitters having different peak wavelengths (e.g., differing by at least 5 nm, at least 10 nm, at least 15 nm, at least 20 nm, or at least 25 nm).

Ultraviolet light (e.g., UV-A light having a peak wavelength in a range of from 350 nm to 395 nm, and UV-B light having a peak wavelength in a range of from 320 nm to 350 nm) may be effective as ES increasing light; however, overexposure to ultraviolet light may lead to detrimental health effects including premature skin aging and potentially elevated risk for certain types of cancer. The combination of light at this wavelength with the anti-inflammatory light can minimize these effects.

In certain embodiments, UV light (e.g., having peak wavelengths in a range of from 320 nm to 399 nm) may be used as ES increasing light; however, in other embodiments, UV light may be avoided.

In certain embodiments, ES increasing light and ES releasing light are substantially free of UV light. In certain embodiments, less than 5% of the ES increasing light is in a wavelength range of less than 400 nm, and less than 1% of the ES releasing light output is in a wavelength range of less than 400 nm. In certain embodiments, ES increasing light includes a peak wavelength in a range of from 400 nm to 490 nm, or from 400 nm to 450 nm, from 400 nm to 435 nm, or from 400 nm to 420 nm.

In certain embodiments, ES increasing light includes a peak wavelength in a range of from 400 nm to 490 nm, or from 400 nm to 450 nm, from 400 nm to 435 nm, or from 400 nm to 420 nm.

In certain embodiments, ES increasing light may include a wavelength range and flux that may alter the presence, concentration, or growth of bacteria or other microbes in or on living mammalian tissue receiving the light. UV light and near-UV light (e.g., having peak wavelengths from 400 nm to 435 nm, or more preferably from 400 nm to 420 nm) in particular may affect microbial growth.

Effects on microbial growth may depend on the wavelength range and dose. In certain embodiments, ES increasing light may include near-UV light having a peak wavelength in a range of from 400 nm to 420 nm to provide a bacterio static effect (e.g., with pulsed light having a radiant flux of <9 mW/cm²), provide a bactericidal effect (e.g., with substantially steady state light having a radiant flux in a range of from 9 mW/cm² to 17 mW/cm²), or provide an antimicrobial effect (e.g., with substantially steady state light having a radiant flux in a range of greater than 17 mW/cm², such as in a range of from 18 mW/cm² to 60 mW/cm²).

In certain embodiments, ES increasing light in a near-UV range (e.g., from 400 nm to 420 nm) may also affect microbial growth (whether in a bacteriostatic range, bactericidal range, or an antimicrobial range) for uses such as wound healing, reduction of acne blemishes, or treatment of atopic dermatitis. Such function(s) may be in addition to the function of the ES increasing light to increase endogenous stores of nitric oxide in living tissue.

In certain embodiments, ES releasing light may include a peak wavelength in a range of from 500 nm to 900 nm, or in a range of from 490 nm to 570 nm, or in a range of from 510 nm to 550 nm, or in a range of from 520 nm to 540 nm, or in a range of from 525 nm to 535 nm, or in a range of from 528 nm to 532 nm, or in a range of about 530 nm.

As shown in U.S. Pat. No. 10,525,275, the wavelengths identified to be most effective in releasing NO from Hb-NO were determined to be the following, from best to worst: 530 nm, 505 nm, 597 nm, 447 nm, 660 nm, 470 nm, 410 nm, 630 nm, and 850 nm.

Wavelengths at 530 nm, 597 nm, 505 nm, 660 nm, 470 nm, 630 nm, 410 nm, 447 nm, and 850 nm released nitric oxide from CCO—NO.

Notably, 530 nm was determined to be the most effective peak wavelength of light for releasing NO from both Hb-NO and CCO—NO.

The wavelength at 660 nm is both anti-inflammatory, and releases NO.

A combination of equal parts of 410 nm light and 530 nm light is equally as effective as 530 nm light alone. Such a combination may be beneficial since a 410 nm blue LED is significantly more efficient than a 530 nm green LED, such that a combination of equal parts of 410 nm LED emissions and 530 nm LED emissions may use 26% less electric power than emissions of a 530 nm LED alone, when operated to provide the same radiant flux.

Light at 660 nm is significantly less effective than the 530 nm green light at releasing NO from Hb-NO. The release of NO from Hb-NO appears to be the same for 530 nm green light, 660 nm red light, and a combination of 530 nm green and 660 nm light for the time window of from 0 seconds to about 2000 seconds, but the effectiveness of the different sources diverges thereafter. Without intending to be bound by any particular theory or explanation of this phenomenon, it is suggested that NO binds to Hb-NO at multiple sites, and that removal of a second or subsequent NO molecule from Hb-NO may require more energy than removal of a first NO molecule, perhaps due to a change in shape of the Hb-NO after removal of a first NO molecule.

In certain embodiments, anti-inflammatory light having a first peak wavelength is impinged on living tissue, and ES increasing or ES releasing light that includes light having a second peak wavelength is impinged on the living tissue, and furthermore a light having a third peak wavelength (i.e., ES releasing or ES increasing light) may be impinged on the living tissue. In certain embodiments, the light having a third peak wavelength may be provided at substantially the same time as (or during a time window overlapping at least one time window of) one or both of the anti-inflammatory and the ES increasing and/or ES releasing light.

In certain embodiments, the light having a third peak wavelength differs from each of the first peak wavelength and the second peak wavelength by at least 10 nm. In certain embodiments, the light having a third peak wavelength exceeds the second peak wavelength by at least 20 nm. In certain embodiments, the light having a third peak wavelength is provided with a radiant flux in a range of from 5 mW/cm² to 60 mW/cm². In certain embodiments, the third peak wavelength is in a range of from 600 nm to 900 nm, or in a range of from 600 nm to 700 nm. In certain embodiments, the third peak wavelength is in a range of from 320 nm to 399 nm.

In certain embodiments, the anti-inflammatory light is in a range of from about 630 nm to 670 nm (e.g., including specific wavelengths of about 630 nm and about 660 nm) may be useful to provide anti-inflammatory effects and/or to promote vasodilation. Anti-inflammatory effects may be useful in treating skin disorders, particularly when combined with ES releasing and/or ES increasing light, to reduce itching, treat psoriasis and other skin disorders, promote wound healing, reduce acne blemishes, promote facial aesthetics, and/or treat atopic dermatitis and other topical dermatological disorders. Vasodilation may also be beneficial to treat androgenic alopecia or other topical dermatological disorders.

Methods of Treatment

Representative skin disorders that can be treated using the methods described herein include pruritus, psoriasis, acne, rosacea, eczema, such as eczema verruca vulgaris, neurofibromatosis, pyogenic granulomas, recessive dystrophic epidermolysis bullosa, venous ulcers, molluscum contagiosum, seborrheic keratosis, Sturge-Weber syndrome, actinic keratosis, and dandruff.

In one embodiment, the skin disorder is pruritus, psoriasis, acne, rosacea, or eczema. In another embodiment, the skin disorder is a disorder of the skin of the scalp, such as pruritis or psoriasis.

In certain embodiments, the anti-inflammatory light may be useful to promote thermal and/or infrared heating of living mammalian tissue, such as may be useful in certain contexts including wound healing.

The methods and devices disclosed herein for treating skin disorders in living mammalian tissue are contemplated for use with a wide variety of tissues. In certain embodiments, the tissue comprises epithelial tissue, which, in some aspects, is tissue of the scalp. In certain embodiments, the tissue comprises mucosal tissue. In certain embodiments, the tissue is within a body cavity of a patient. In certain embodiments, the tissue comprises cervical tissue.

Devices

There is no particular limit on the types of device used to deliver light at anti-inflammatory and ES increasing and/or ES releasing wavelengths, so long as the appropriate wavelengths of light can be delivered at an appropriate flux, and for an appropriate time, to treat the skin disorder.

In some embodiments, the devices will be in the form of a flexible bandage equipped with the ability to emit light at the desired wavelengths.

In some embodiments, the devices will be in the form of skin plasters or masks.

In still other embodiments, the devices will be in the form of hand-held light-emitting “wands.”

In some embodiments, particularly when the devices are used to treat skin disorders of the scalp, the devices can be in the form of a helmet, cap, or other device adapted for applying light to the scalp.

In certain aspects of the latter embodiment, a device for treating skin disorders in living mammalian tissue as disclosed herein may include a flexible substrate supporting one or more light emitting elements and arranged to conform to at least a portion of a human body. In certain embodiments, a flexible substrate may include a flexible printed circuit board (PCB), such as may include at least one polyimide-containing layer and at least one layer of copper or another electrically conductive material.

In other embodiments, a device for treating skin disorders as disclosed herein may include a rigid substrate supporting one or more light emitting elements. In certain embodiments, one or more surfaces of a device for for treating skin disorders may include a light-transmissive encapsulant material arranged to cover any light emitter(s) and at least a portion of an associated substrate (e.g., flexible PCB). A preferred encapsulant material is silicone, which may be applied by any suitable means such as molding, dipping, spraying, dispensing, or the like. In certain embodiments, one or more functional materials may be added to or coated on an encapsulant material. In certain embodiments, at least one surface, or substantially all surfaces (e.g., front and back surfaces) of a flexible PCB may be covered with encapsulant material.

In certain embodiments, a substrate as described herein may be arranged to support one or more light emitting elements. In certain embodiments, one or more light emitting elements may include multi-emitting light emitting devices such as multi-LED packages. In certain embodiments, one or more light emitting elements may be arranged for direct illumination, wherein at least a portion of emissions generated by light emitting element are arranged to be transmitted directly through a light-transmissive external surface of a device without need for an intervening waveguide or reflector. In certain embodiments, one or more light emitting elements may be arranged for indirect illumination (e.g., side illumination), wherein emissions generated by light emitting element are arranged to be transmitted to a light-transmissive external surface via a waveguide and/or a reflector, without a light emitting element being in direct line-of-sight arrangement relative to a light-transmissive external surface. In certain embodiments, a hybrid configuration may be employed, including one or more light emitting elements arranged for direct illumination, and further including one or more light emitting elements arranged for indirect illumination. In certain embodiments, one or more reflective materials (e.g., reflective flexible PCB or other reflective films) may be provided along selected surfaces of a device to reduce internal absorption of light and to direct light emissions toward an intended light-transmissive surface. In certain embodiments, a flexible light emitting device may include a substantially uniform thickness. In other embodiments, a flexible light emitting device may include a thickness that varies with position, such as a thickness that tapers in one direction or multiple directions. In certain embodiments, presence of a tapered thickness may help a flexible light emitting device to more easily be wrapped against or to conform to areas of a mammalian (e.g., human) body.

In certain embodiments, one or multiple holes or perforations may be defined in a substrate and any associated encapsulant material. In certain embodiments, holes may be arranged to permit transit of air, such as may be useful for thermal management. In certain embodiments, holes may be arranged to permit transit of wound exudate. In certain embodiments, one or more holes may be arranged to permit sensing of at least one condition through the hole(s). Holes may be defined by any suitable means such as laser perforation, die pressing, slitting, punching, blade cutting, and roller perforation. In certain embodiments, holes may have uniform or non-uniform size, placement, and/or distribution relative to a substrate and encapsulant material.

In certain embodiments, a device for for treating skin disorders as disclosed herein may include one or more light-affecting elements such as one or more light extraction features, wavelength conversion materials, light diffusion or scattering materials, and/or light diffusion or scattering features. In certain embodiments, one or more light affecting elements may be arranged in a layer between a light emitting element and a light transmissive surface of a device. In certain embodiments, an encapsulant material (e.g., encapsulant material layer) may be arranged between at least one light emitting element and one or more light affecting elements. In certain embodiments, one or more light affecting elements may be formed or dispersed within an encapsulant material.

In certain embodiments, impingement of light on living tissue and/or operation of a device as disclosed herein may be responsive to one or more signals generated by one or more sensors or other elements. Various types of sensors are contemplated, including temperature sensors, photosensors, image sensors, proximity sensors, pressure sensors, chemical sensors, biosensors, accelerometers, moisture sensors, oximeters, current sensors, voltage sensors, and the like. Other elements that may affect impingement of light and/or operation of a device as disclosed herein include a timer, a cycle counter, a manually operated control element, a wireless transmitter and/or receiver (as may be embodied in a transceiver), a laptop or tablet computer, a mobile phone, or another portable digital device. Wired and/or wireless communication between a device as disclosed herein and one or more signal generating or signal receiving elements may be provided.

In certain embodiments, impingement of light on living tissue and/or operation of a device as disclosed herein may be responsive to one or more temperature signals. For example, a temperature condition may be sensed on or proximate to (a) a device arranged to emit ES generating light and/or ES releasing light or (b) the tissue; at least one signal indicative of the temperature condition may be generated; and operation of a lighting device may be controlled responsive to the at least one signal. Such control may include initiation of operation, deviation (or alteration) of operation, or termination of operation of light emitting elements, such as elements arranged to emit anti-inflammatory and ES generating light and/or ES releasing light. In certain embodiments, thermal foldback protection may be provided at a threshold temperature (e.g., >42° Celsius) to prevent a user from experiencing burns or discomfort. In certain embodiments, thermal foldback protection may trigger a light emitting device to terminate operation, reduce current, or change an operating state in response to receipt of a signal indicating an excess temperature condition.

In certain embodiments, a device for treating skin disorders as disclosed herein may be used for wound care, and may include one or more sensors. In certain embodiments, one or more light emitters and photodiodes may be provided to illuminate a wound site with one or more selected wavelengths to detect blood flow in or proximate to the wound site to provide photoplethsmyography data. One sensor or multiple sensors may be provided. A device may alternatively or additionally include sensors arranged to detect blood pressure, bandage or dressing covering pressure, heart rate, temperature, presence or concentration of chemical or biological species (e.g., in wound exudate), or other conditions.

In certain embodiments, a device for treating skin disorders as disclosed herein may include a memory element to store information indicative of one or more sensor signals. Such information may be used for diagnosis, assessing patient compliance, assessing patient status, assessing patient improvement, and assessing function of the device. In certain embodiments, information indicative of one or more sensor signals may be transmitted via wired or wireless means (e.g., via Bluetooth, WiFi, Zigbee, or another suitable protocol) to a mobile phone, a computer, a data logging device, or another suitable device that may optionally be connected to a local network, a wide-area network, a telephonic network, or other communication network. In certain embodiments, a data port (e.g., micro USB or other type) may be provided to permit extraction or interrogation of information contained in a memory.

Details of illustrative devices that may be used for modulating nitric oxide in living mammalian tissue are described hereinafter.

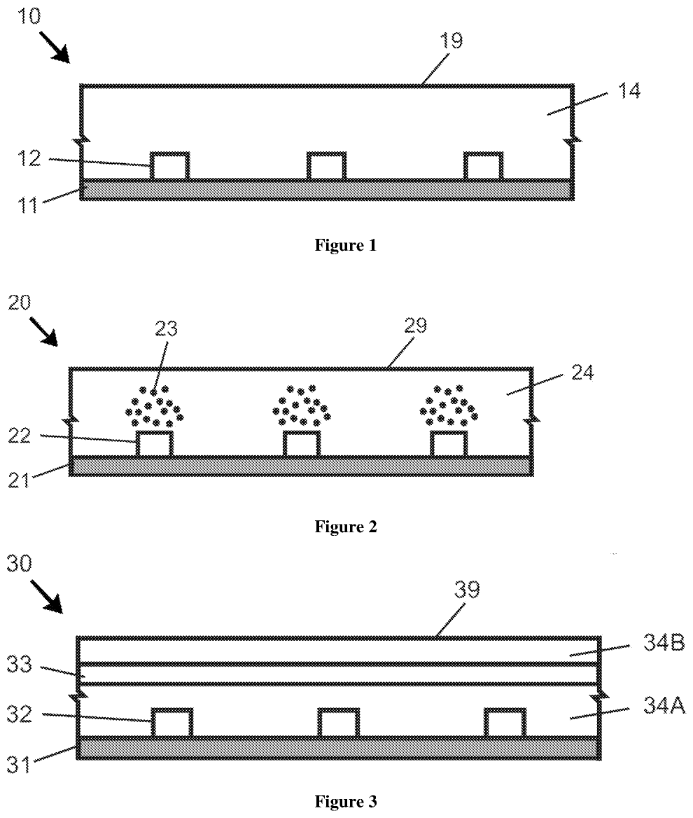

FIG. 1 is a side cross-sectional schematic view of a portion of a device 10 for delivering light energy to living mammalian tissue, the device 10 including multiple direct view light emitting sources 12 supported by a substrate 11 and covered with an encapsulant material 14, which may be embodied in a sheet or layer. The substrate 11 preferably includes a flexible PCB, which may include a reflective surface to reflect light toward a light-transmissive outer surface 19 of the device 10. As shown in FIG. 1, the encapsulant material 14 covers the light emitting sources 12 and an upper surface of the substrate 11; however, it is to be appreciated that in certain embodiments the encapsulant material 14 may cover both upper and lower surfaces of the substrate 11. In certain embodiments, different light emitting sources 12 may generate light having different peak wavelengths. In certain embodiments, one or more light emitting sources 12 may include a multi-emitter package arranged to generate one or multiple peak wavelengths of light. In certain embodiments, one or more light emitting sources 12 may be arranged to produce one or both of anti-inflammatory and ES increasing light or ES releasing light.

FIG. 2 is a side cross-sectional schematic view of a portion of a device 20 for delivering light energy to living mammalian tissue, the device 20 including multiple direct view light emitting sources 22 supported by a substrate 21 and covered with an encapsulant material 24, which may be embodied in a sheet or layer. The substrate 21 preferably includes a flexible PCB, which may include a reflective surface to reflect light toward a light-transmissive outer surface 29 of the device 20. At least one functional material (e.g., wavelength conversion material and/or scattering material) 23 is disposed within the encapsulant material 24. In certain embodiments, the functional material(s) 23 include one or more wavelength conversion materials, such as at least one of a phosphor material, a fluorescent dye material, a quantum dot material, and a fluorophore material. In certain embodiments, wavelength materials of different peak wavelengths may be applied over different light emitting sources 22. In certain embodiments, the functional material(s) 23 are applied by dispensing or printing. In certain embodiments, one or more light emitting sources 22 may include a multi-emitter package arranged to generate one or multiple peak wavelengths of light. In certain embodiments, one or more light emitting sources 22 may be arranged to produce one or both of anti-inflammatory and ES increasing light or ES releasing light.

FIG. 3 is a side cross-sectional schematic view of a portion of a device 30 for delivering light energy to living mammalian tissue, the device 30 including multiple direct view light emitting sources 32 supported by a substrate 31 and covered with two encapsulant material layers 34A, 34B, with at least one functional material (e.g., wavelength conversion and/or scattering material) sheet or layer 33 disposed between the encapsulant material layers 34A, 34B. The substrate 31 preferably includes a flexible PCB, which may include a reflective surface to reflect light toward a light-transmissive outer surface 39 of the device 30. In certain embodiments, the functional material(s) 33 include one or more wavelength conversion materials, such as at least one of a phosphor material, a fluorescent dye material, a quantum dot material, and a fluorophore material. In certain embodiments, one or more light emitting sources 32 may include a multi-emitter package arranged to generate one or multiple peak wavelengths of light. In certain embodiments, one or more light emitting sources 32 may be arranged to produce one or both of anti-inflammatory and ES increasing light or ES releasing light.

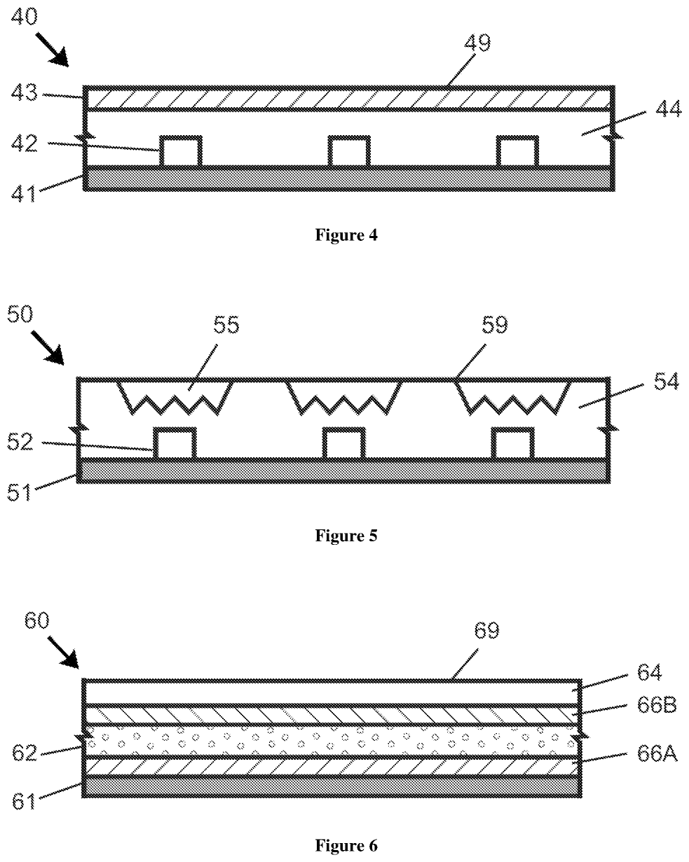

FIG. 4 is a side cross-sectional schematic view of a portion of a device 40 for delivering light energy to living mammalian tissue, the device 40 including multiple direct view light emitting sources 42 supported by a substrate 41 and covered by an encapsulant material 44, which may be embodied in a sheet or layer. The substrate 41 preferably includes a flexible PCB, which may include a reflective surface to reflect light toward a light-transmissive outer surface 49 of the device 40. The encapsulant material 44 is covered with a diffusion or scattering material layer 49. In certain embodiments, the diffusion or scattering material layer 49 may include acrylic, PET, silicone, or a polymeric sheet. In certain embodiments, the diffusion or scattering material layer 49 may include scattering particles such as zinc oxide, silicon dioxide, titanium dioxide, or the like. In certain embodiments, one or more light emitting sources 42 may include a multi-emitter package arranged to generate one or multiple peak wavelengths of light. In certain embodiments, one or more light emitting sources 42 may be arranged to produce one or both of anti-inflammatory and ES increasing light or ES releasing light.

FIG. 5 is a side cross-sectional schematic view of a portion of a device 50 for delivering light energy to living mammalian tissue, the device 50 including multiple direct view light emitting sources 52 supported by a substrate 51. The substrate 51 preferably includes a flexible PCB, which may include a reflective surface to reflect light toward a light-transmissive outer surface 59 of the device 50. Multiple molded features 55 (e.g., molded from silicone) overlie the light emitting sources 52. An encapsulant or light coupling material 54 is arranged between the light emitting sources 52 and the molded features 55. In certain embodiments, light coupling material 54 may include a light coupling gel with an index of refraction that differs from an index of refraction of the molded features 55. The molded features 55 may be arranged along a light transmissive outer surface 59 of the device 50. In certain embodiments, one or more light emitting sources 52 may include a multi-emitter package arranged to generate one or multiple peak wavelengths of light. In certain embodiments, one or more light emitting sources 52 may be arranged to produce one or both of ES increasing light and ES releasing light.

FIG. 6 is a side cross-sectional schematic view of a portion of a device 60 for delivering light energy to living mammalian tissue, the device 60 including a flexible substrate 61, a passive-matrix organic light emitting diode (OLED) structure (embodied in an anode layer 66A, a cathode layer 66B, and an OLED stack 62 between the anode and cathode layers 66A, 66B. In certain embodiments, the OLED stack 62 may be configured to generate multiple wavelengths of light. The substrate 61 preferably includes a flexible PCB, which may include a reflective surface to reflect light toward a light-transmissive outer surface 69 of the device 60. An encapsulant layer 64 is arranged over the cathode layer 66B and preferably defines an outer light-transmissive surface 69 of the device 60. In certain embodiments, one or more light emitting wavelengths produced by the OLED stack 62 may include anti-inflammatory and ES increasing light and/or ES releasing light.

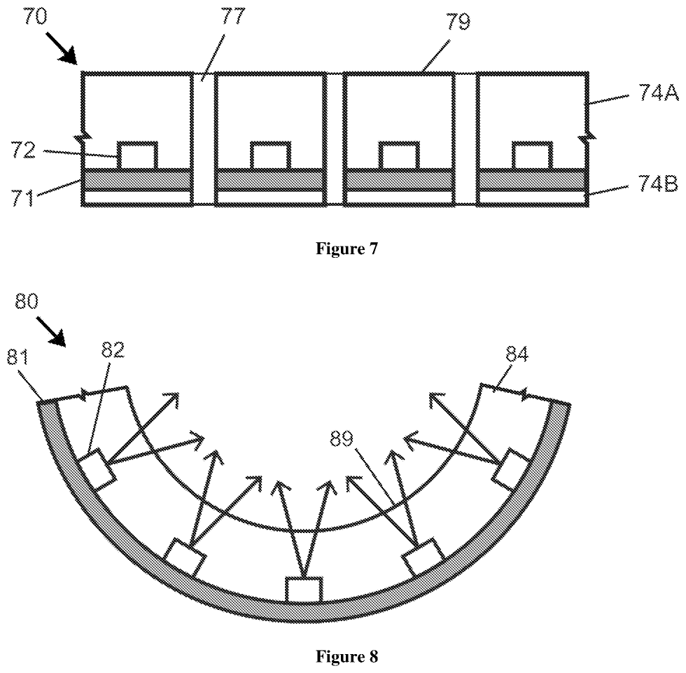

FIG. 7 is a side cross-sectional schematic view of a portion of a device 70 for delivering light energy to living mammalian tissue, the device 70 including a flexible substrate 71, multiple direct view light emitting sources 72 supported by the substrate 71, and encapsulant material layers 74A, 74B arranged above and below the substrate, respectively. The substrate 71 preferably includes a flexible PCB, which may include a reflective surface to reflect light toward a light-transmissive outer surface 79 of the device 70. The light emitting device 70 further includes holes or perforations 77 defined through both the substrate 71 and the encapsulant material layers 74A, 74B. In certain embodiments, one or more light emitting sources 72 may be arranged to produce one or both of anti-inflammatory and ES increasing light or ES releasing light.

FIG. 8 is a side cross-sectional schematic view of a portion of a device 80 for delivering light energy to living mammalian tissue, wherein the device 80 includes multiple direct view light emitting sources 82 supported by a flexible substrate 81 and covered by an encapsulant layer 84. The substrate 81 preferably includes a flexible PCB, which may include a reflective surface to reflect light toward a light-transmissive outer surface 89 of the device 80. The device 80 is preferably flexible to permit it to be bent or shaped into a variety of shapes to conform to a portion of a mammalian body. As illustrated, the device 80 is arranged in a concave configuration with the multiple light emitting sources 82 arranged to direct emissions toward a center of curvature of the device 80. In certain embodiments, one or more light emitting sources 82 may be arranged to produce one or both of anti-inflammatory and ES increasing light or ES releasing light.

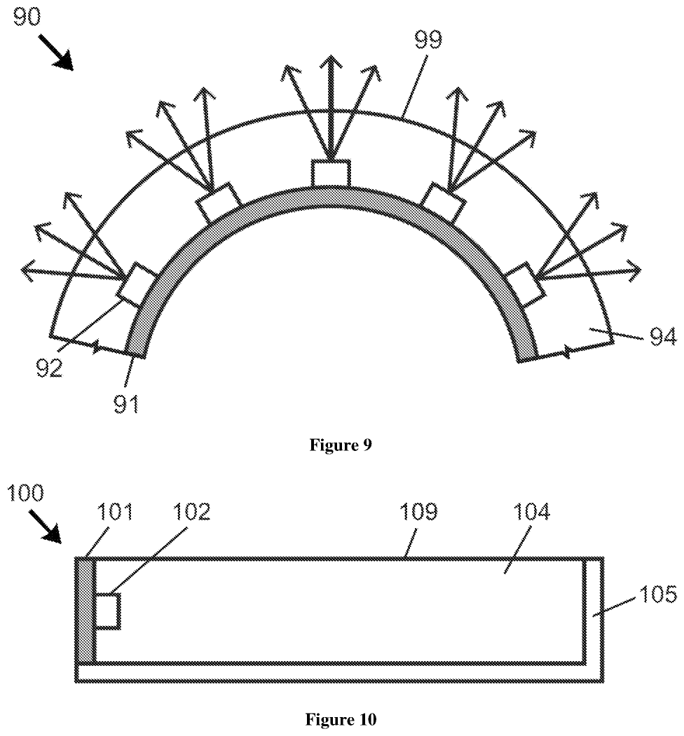

FIG. 9 is a side cross-sectional schematic view of a portion of a device 90 for delivering light energy to living mammalian tissue, wherein the device 90 includes multiple direct view light emitting sources 92 supported by a flexible substrate 91 and covered by an encapsulant layer 94. The substrate 91 preferably includes a flexible PCB, which may include a reflective surface to reflect light toward a light-transmissive outer surface 99 of the device 90. The device 90 is preferably flexible to permit it to be bent or shaped into a variety of shapes to conform to a portion of a mammalian body. As illustrated, the device 90 is arranged in a convex configuration with the multiple light emitting elements 92 arranged to direct emissions away from a center of curvature of the device 90. In certain embodiments, one or more light emitting sources 92 may be arranged to produce one or both of anti-inflammatory and ES increasing light or ES releasing light.

FIG. 10 is a side cross-sectional schematic view of a portion of a device 100 for delivering light energy to living mammalian tissue, wherein the device 100 is edge lit with one or more light emitting sources 102 supported by a flexible printed circuit board (PCB) 101 that preferably includes a reflective surface. Other non-light-transmitting surfaces of the device 100 are bounded by a flexible reflective substrate 105 arranged to reflect light toward a light-transmissive outer surface 109 of the device 100. The flexible PCB 101, the light emitting source(s) 102, and the flexible reflective substrate 105 are covered with an encapsulant material 104, which may include silicone. As illustrated, the device 100 may include a substantially constant thickness. In certain embodiments, one or more light emitting sources 102 may be arranged to produce one or both of anti-inflammatory and ES increasing light or ES releasing light.

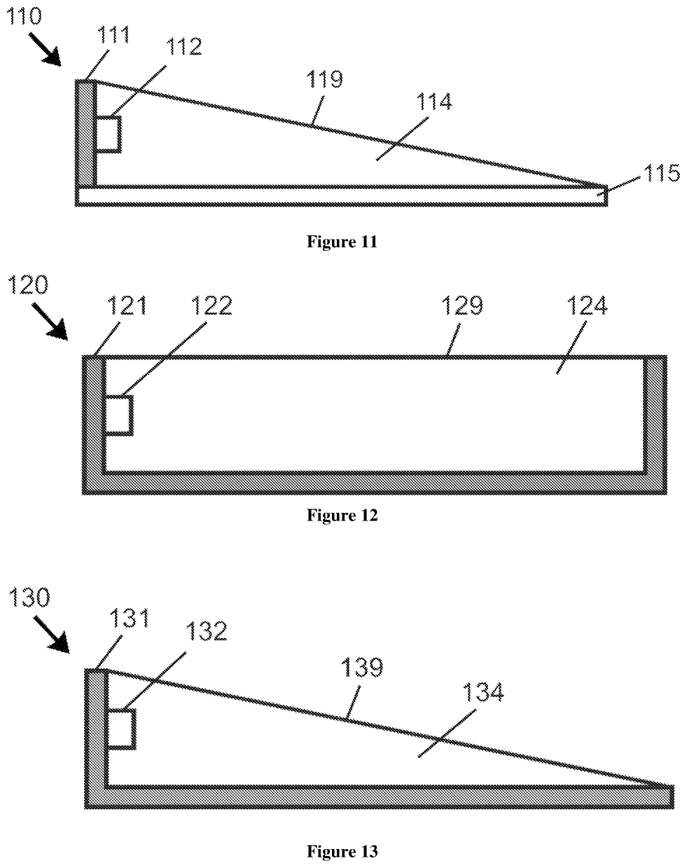

FIG. 11 is a side cross-sectional schematic view of a portion of a device 110 for delivering light energy to living mammalian tissue, wherein the device 110 is edge lit with one or more light emitting sources 112 supported by a flexible PCB 111 that preferably includes a reflective surface. A non-light-transmitting face of the device 110 is bounded by a flexible reflective substrate 115 arranged to reflect light toward a light-transmissive outer surface 119 of the device 110. The flexible PCB 111, the light emitting source(s) 112, and the flexible reflective substrate 115 are covered with an encapsulant material 114, which may include silicone. As illustrated, the device 110 may include a thickness that is tapered with distance away from the light emitting sources 112. Such tapered thickness may enable the device 110 to more easily be wrapped against or to conform to areas of a mammalian (e.g., human) body. In certain embodiments, one or more light emitting sources 112 may be arranged to produce one or both of anti-inflammatory and ES increasing light or ES releasing light.

FIG. 12 is a side cross-sectional schematic view of a portion of a device 120 for delivering light energy to living mammalian tissue, wherein the device 120 is edge lit with one or more light emitting sources 122 supported by a flexible PCB 121 that bounds multiple edges and a face of the device 120. The flexible PCB 121 preferably includes a reflective surface arranged to reflect light toward a light-transmissive outer surface 129 of the device 120. The flexible PCB 121 and the light emitting source(s) 122 are covered with an encapsulant material 124, which may include silicone. In certain embodiments, one or more light emitting sources 122 may be arranged to produce one or both of anti-inflammatory and ES increasing light or ES releasing light.

FIG. 13 is a side cross-sectional schematic view of a portion of a device 130 for delivering light energy to living mammalian tissue, wherein the device 130 is edge lit with one or more light emitting sources 132 supported by a flexible PCB 131 that bounds one edge and one face of the device 130. The flexible PCB 131 preferably includes a reflective surface arranged to reflect light toward a light-transmissive outer surface 139 of the device 130. The flexible PCB 131 and the light emitting source(s) 132 are covered with an encapsulant material 134, which may include silicone. As illustrated, the device 130 may include a thickness that is tapered with distance away from the light emitting sources 132. In certain embodiments, one or more light emitting sources 132 may be arranged to produce one or both of anti-inflammatory and ES increasing light or ES releasing light.

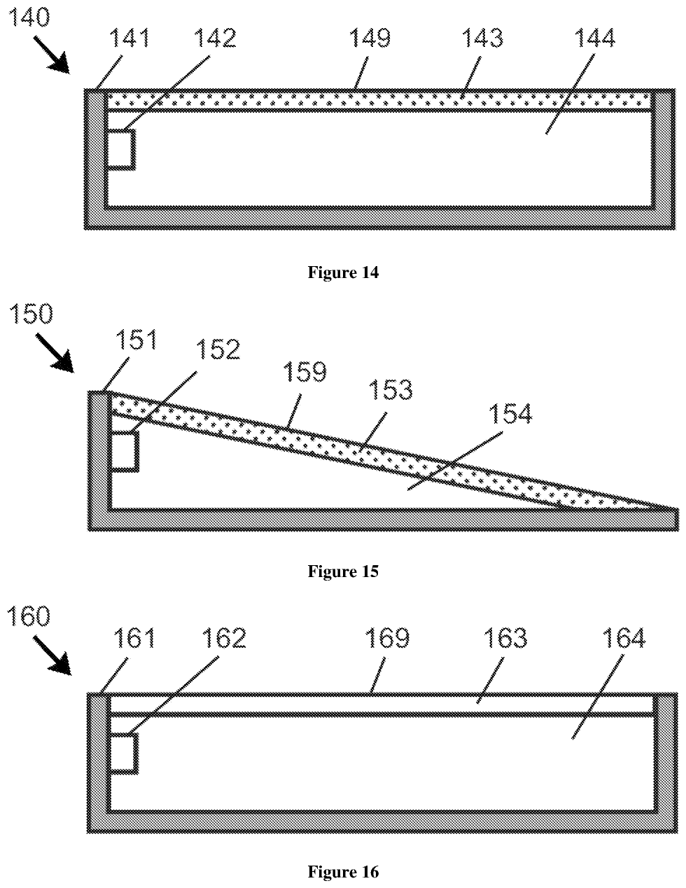

FIG. 14 is a side cross-sectional schematic view of a portion of a device 140 for delivering light energy to living mammalian tissue, wherein the device 140 is edge lit with one or more light emitting sources 142 supported by a flexible PCB 141 that bounds multiple edges and a face of the device 140. In certain embodiments, one or more light emitting sources 142 may include a multi-emitter package arranged to generate one or multiple peak wavelengths of light. The flexible PCB 141 preferably includes a reflective surface arranged to reflect light toward a light-transmissive outer surface 149 of the device 140. The flexible PCB 141 and the light emitting source(s) 142 are covered with an encapsulant material 144, which may include silicone. Between the light-transmitting outer surface 149 and the encapsulant material 144, the device 140 further includes a diffusing and/or scattering layer 143. In certain embodiments, the diffusing and/or scattering layer 143 may include a sheet of material; in other embodiments, the diffusing and/or scattering layer 143 may include particles applied in or on the encapsulant material 144. In certain embodiments, one or more light emitting sources 142 may be arranged to produce one or both of anti-inflammatory and ES increasing light or ES releasing light.

FIG. 15 is a side cross-sectional schematic view of a portion of a device 150 for delivering light energy to living mammalian tissue, wherein the device 150 is edge lit with one or more light emitting sources 152 supported by a flexible PCB 151 that bounds one edge and one face of the device 150. In certain embodiments, one or more light emitting sources 152 may include a multi-emitter package arranged to generate one or multiple peak wavelengths of light. The flexible PCB 151 preferably includes a reflective surface arranged to reflect light toward a light-transmissive outer surface 159 of the device 150. The flexible PCB 151 and the light emitting source(s) 152 are covered with an encapsulant material 154, which may include silicone. Between the light-transmitting outer surface 159 and the encapsulant material 154, the device 150 further includes a diffusing and/or scattering layer 153. In certain embodiments, the diffusing and/or scattering layer 153 may include a sheet of material; in other embodiments, the diffusing and/or scattering layer 153 may include particles applied in or on the encapsulant material 154. As illustrated, the device 150 may include a thickness that is tapered with distance away from the light emitting sources 152. In certain embodiments, one or more light emitting sources 152 may be arranged to produce one or both of anti-inflammatory and ES increasing light or ES releasing light.

FIG. 16 is a side cross-sectional schematic view of a portion of a device 160 for delivering light energy to living mammalian tissue, wherein the device 160 is edge lit with one or more light emitting sources 162 supported by a flexible PCB 161 that bounds multiple edges and a face of the device 160. In certain embodiments, one or more light emitting sources 162 may include a multi-emitter package arranged to generate one or multiple peak wavelengths of light. The flexible PCB 161 preferably includes a reflective surface arranged to reflect light toward a light-transmissive outer surface 169 of the device 160. The flexible PCB 161 and the light emitting source(s) 162 are covered with an encapsulant material 164, which may include silicone. Between the light-transmitting outer surface 169 and the encapsulant material 164, the device 160 further includes a wavelength conversion material 163. In certain embodiments, the wavelength conversion material 163 may include a sheet or layer of material; in other embodiments, the wavelength conversion material 163 may include particles applied in or on the encapsulant material 164. In certain embodiments, one or more light emitting sources 162 may be arranged to produce one or both of anti-inflammatory and ES increasing light or ES releasing light.

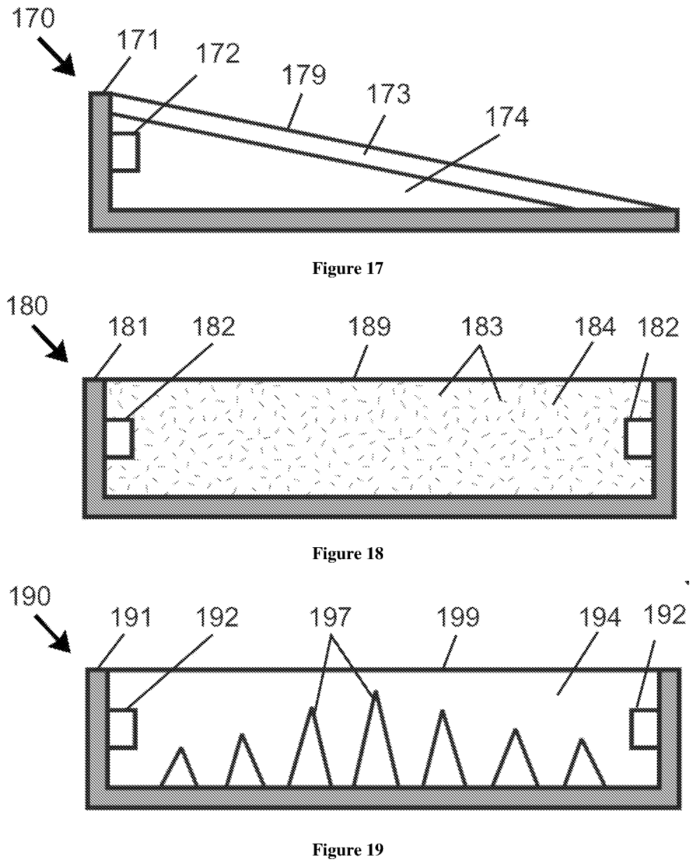

FIG. 17 is a side cross-sectional schematic view of a portion of a device 170 for delivering light energy to living mammalian tissue, wherein the device 170 is edge lit with one or more light emitting sources 172 supported by a flexible PCB 171 that bounds one edge and one face of the device 170. In certain embodiments, one or more light emitting sources 172 may include a multi-emitter package arranged to generate one or multiple peak wavelengths of light. The flexible PCB 171 preferably includes a reflective surface arranged to reflect light toward a light-transmissive outer surface 179 of the device 170. The flexible PCB 171 and the light emitting source(s) 172 are covered with an encapsulant material 174, which may include silicone. Between the light-transmitting outer surface 179 and the encapsulant material 174, the device 170 further includes a wavelength conversion material 173. In certain embodiments, the wavelength conversion material 173 may include a sheet or layer of material; in other embodiments, the wavelength conversion material 173 may include particles applied in or on the encapsulant material 174. As illustrated, the device 170 may include a thickness that is tapered with distance away from the light emitting sources 172. In certain embodiments, one or more light emitting sources 172 may be arranged to produce one or both of anti-inflammatory and ES increasing light or ES releasing light.

FIG. 18 is a side cross-sectional schematic view of a portion of a device 180 for delivering light energy to living mammalian tissue, wherein the device 180 is edge lit along multiple edges with multiple light emitting sources 182 supported by a flexible PCB 181 having a reflective surface arranged to reflect light toward a light-transmissive outer surface 189 of the device 180. The flexible PCB 181 and light emitting sources 182 are covered with an encapsulant material 184, and a wavelength conversion material 183 is distributed in the encapsulant material 184. In certain embodiments, one or more light emitting sources 182 may include a multi-emitter package arranged to generate one or multiple peak wavelengths of light. In certain embodiments, one or more light emitting sources 182 may be arranged to produce one or both of anti-inflammatory and ES increasing light or ES releasing light.

FIG. 19 is a side cross-sectional schematic view of a portion of a device 190 for delivering light energy to living mammalian tissue, wherein the device 190 is edge lit along multiple edges with multiple light emitting sources 192 supported by a flexible PCB 191 having a reflective surface arranged to reflect light toward a light-transmissive outer surface 199 of the device 190. The device 190 further includes raised light extraction features 197 supported by the flexible PCB 191, with such features 197 serving to reflect laterally-transmitted light toward the outer surface 199. An encapsulant material 194 is provided over the flexible PCB 191, the light emitting sources 192, and the light extraction features 197. In certain embodiments, one or more light emitting sources 192 may include a multi-emitter package arranged to generate one or multiple peak wavelengths of light. In certain embodiments, one or more light emitting sources 192 may be arranged to produce one or both of anti-inflammatory and ES increasing light or ES releasing light.

In certain embodiments, the light extraction features 197 may be dispensed, molded, layered, or painted on the flexible PCB 191. In certain embodiments, different light extraction features 197 may include different indices of refraction. In certain embodiments, different light extraction features 197 may include different sizes and/or shapes. In certain embodiments, light extraction features 197 may be uniformly or non-uniformly distributed over the flexible PCB 191. In certain embodiments, light extraction features 197 may include tapered surfaces. In certain embodiments, different light extraction features 197 may include one or more connected portions or surfaces. In certain embodiments, different light extraction features 197 may be discrete or spatially separated relative to one another. In certain embodiments, light extraction features 197 may be arranged in lines, rows, zig-zag shapes, or other patterns. In certain embodiments, one or more wavelength conversion materials may be arranged on or proximate to one or more light extraction features 197.

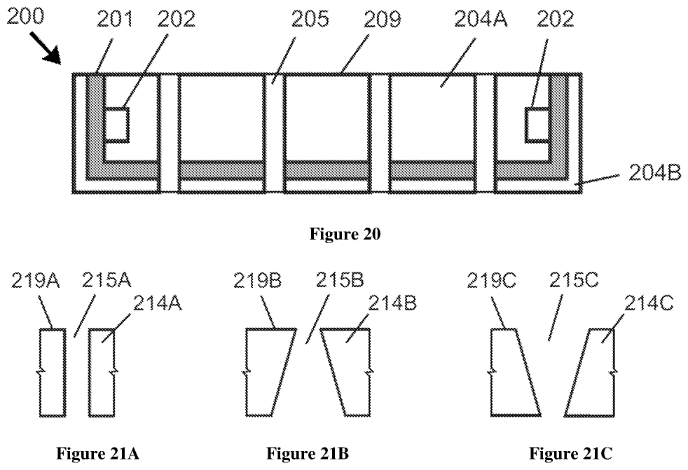

FIG. 20 is a side cross-sectional schematic view of a portion of a device 200 for delivering light energy to living mammalian tissue, wherein the device 200 is edge lit along multiple edges with multiple light emitting sources 202 supported by a flexible PCB 201 having a reflective surface arranged to reflect light toward a light-transmissive outer surface 209 of the device 200. In certain embodiments, one or more light emitting sources 202 may be arranged to produce one or both of anti-inflammatory and ES increasing light or ES releasing light. Encapsulant material layers 204A, 204B are arranged above and below the PCB 201 and over the light emitting sources 202. Holes or perforations 205 are defined through the substrate 201 and the encapsulant material layers 204A, 204B. The holes or perforations 205 preferably allow passage of at least one of air and exudate through the device 200.

Holes or perforations defined through a device (e.g., through a PCB and encapsulant layers) as described herein may include holes of various shapes and configurations. Holes may be round, oval, rectangular, square, polygonal, or any other suitable axial shape. Cross-sectional shapes of holes or perforations may be constant or non-constant. Cross-sectional shapes that may be employed according to certain embodiments are shown in FIGS. 21A-21C.

FIG. 21A is a cross-sectional view of a first exemplary hole 215A definable through an encapsulant layer 214A of a device for delivering light energy to living mammalian tissue, the hole 215A having a diameter that is substantially constant with depth and extending to an outer light transmitting surface 219A.

FIG. 21B is a cross-sectional view of a second exemplary hole 215B definable through an encapsulant layer 214B of a device for delivering light energy to living mammalian tissue, the hole 215B having a diameter that increases with increasing depth and extending to an outer light transmitting surface 219B. FIG. 21C is a cross-sectional view of a second exemplary hole 215C definable through an encapsulant layer 214C of a device for delivering light energy to living mammalian tissue, the hole 215C having a diameter that decreases with increasing depth and extending to an outer light transmitting surface 219C.

In certain embodiments, perforations or holes may encompass at least 2%, at least 5%, at least 7%, at least 10%, at least 15%, at least 20%, or at least 25% of a facial area of a device for delivering light energy to living mammalian tissue as disclosed herein. In certain embodiments, one or more of the preceding ranges may be bounded by an upper limit of no greater than 10%, no greater than 15%, no greater than 20%, or no greater than 30%. In certain embodiments, perforations or holes may be provided with substantially uniform size and distribution, with substantially uniform distribution but non-uniform size, with non-uniform size and non-uniform distribution, or any other desired combination of size and distribution patterns.

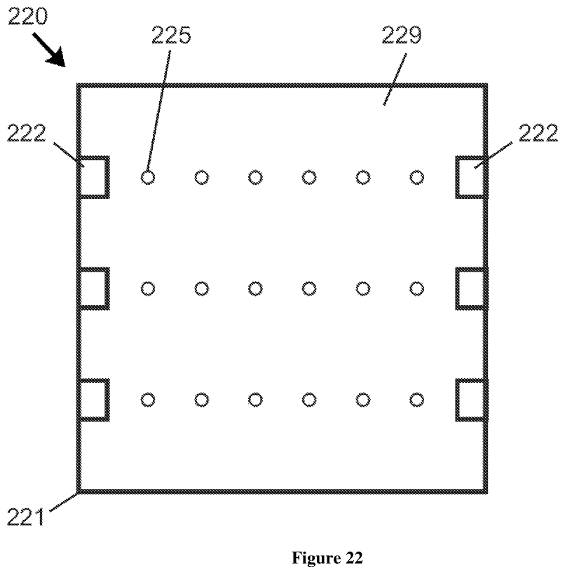

FIG. 22 is a top schematic view of at least a portion of a device 220 for delivering light energy to living mammalian tissue, wherein the device 220 is edge lit along multiple edges with multiple light emitting sources 222 supported by a flexible PCB 221. The PCB 221 is preferably encapsulated on one or both sides with an encapsulant material. Multiple holes or perforations 225 of substantially uniform size and substantially uniform distribution are defined through the flexible PCB 221 and any associated encapsulant material layers. The flexible PCB 221 preferably includes a reflective material arranged to reflect light toward a light transmissive outer surface 229 of the device 220. In certain embodiments, one or more light emitting sources 222 may be arranged to produce one or both of anti-inflammatory and ES increasing light or ES releasing light.

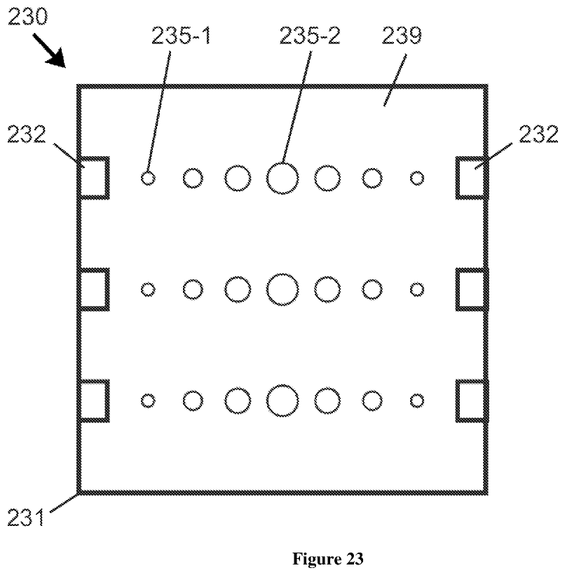

FIG. 23 is a top schematic view of at least a portion of a device 230 for delivering light energy to living mammalian tissue, wherein the device 230 is edge lit along multiple edges with multiple light emitting sources 232 supported by a flexible PCB 231. The PCB 231 is preferably encapsulated on one or both sides with an encapsulant material. Multiple holes or perforations 235-1, 235-2 of differing sizes, but substantially uniform distribution, are defined through the flexible PCB 231 and any associated encapsulant material layers. The flexible PCB 231 preferably includes a reflective material arranged to reflect light toward a light transmissive outer surface 239 of the device 230. In certain embodiments, one or more light emitting sources 232 may be arranged to produce one or both of anti-inflammatory and ES increasing light or ES releasing light.

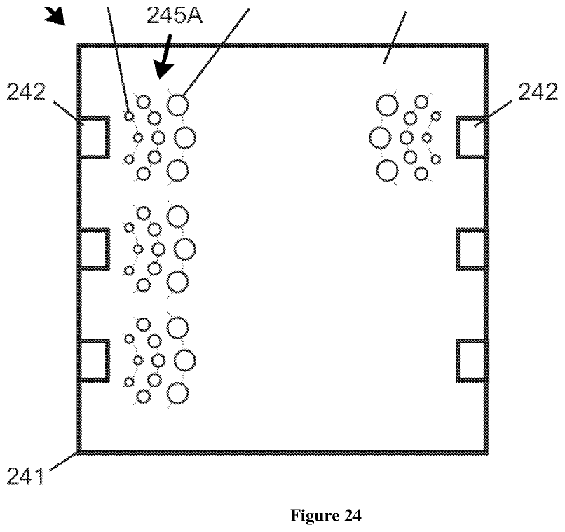

FIG. 24 is a top schematic view of at least a portion of a device 240 for delivering light energy to living mammalian tissue, wherein the device 240 is edge lit along multiple edges with multiple light emitting sources 242 supported by a flexible PCB 241. The PCB 241 is preferably encapsulated on one or both sides with an encapsulant material. The flexible PCB 241 preferably includes a reflective material arranged to reflect light toward a light transmissive outer surface 249 of the device 240. Multiple holes or perforations 245-1, 245-2 of different sizes are provided in one or more clusters 245A (e.g., proximate to one or more light emitting sources 242) and defined through the flexible PCB 241 and any associated encapsulant material layers. In certain embodiments, one or more light emitting sources 242 may be arranged to produce one or both of anti-inflammatory and ES increasing light or ES releasing light.

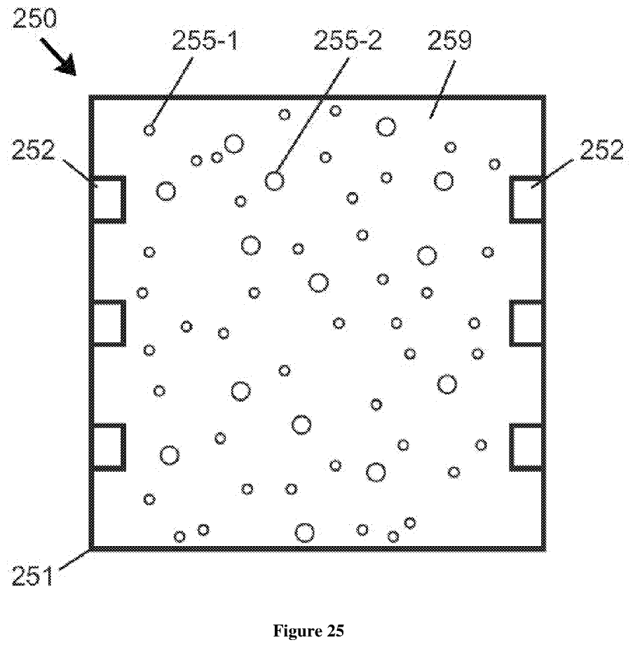

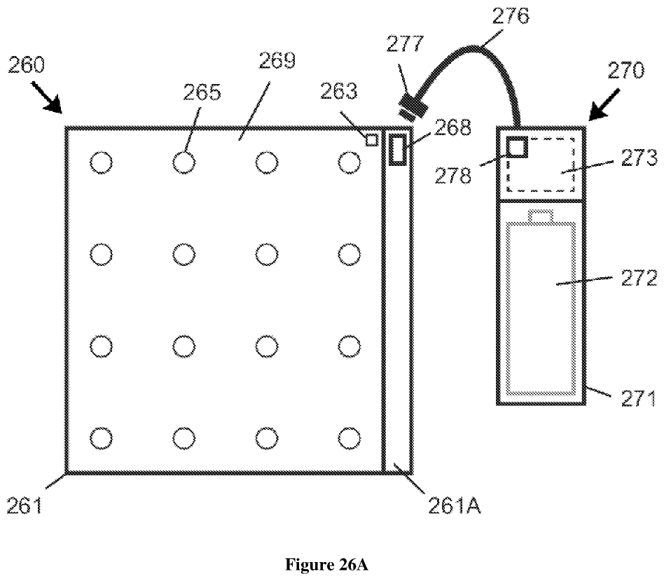

FIG. 25 is a top schematic view of at least a portion of a device 250 for delivering light energy to living mammalian tissue, wherein the device 250 is edge lit along multiple edges with multiple light emitting sources 252 supported by a flexible PCB 251. The PCB 251 is preferably encapsulated on one or both sides with an encapsulant material. The flexible PCB 251 preferably includes a reflective material arranged to reflect light toward a light transmissive outer surface 259 of the device 250. Multiple holes or perforations 255-1, 255-2 of different sizes and with a non-uniform (e.g., random) distribution are defined through the flexible PCB 251 and any associated encapsulant material layers. In certain embodiments, one or more light emitting sources 252 may be arranged to produce one or both of anti-inflammatory and ES increasing light or ES releasing light.

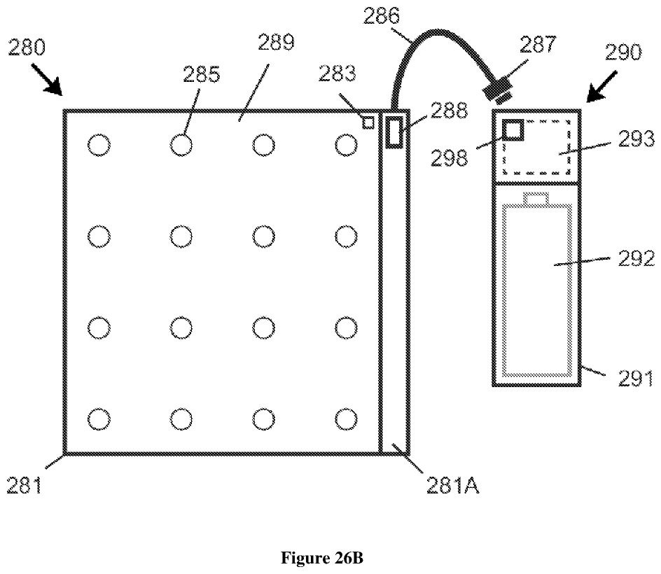

FIG. 26A is a top schematic view of at least a portion of a light emitting device 260 for delivering light energy to living mammalian tissue and at least a portion of a battery/control module 270, wherein an elongated electrical cable 276 is associated with the battery/control module 270 for connecting the battery/control module 270 to the light emitting device 260. The light emitting device 260 is edge lit along one edge with a light emitting region 261A supported by a flexible PCB 261. The PCB 261 is preferably encapsulated on one or both sides with an encapsulant material. The flexible PCB 261 preferably includes a reflective material arranged to reflect light toward a light transmissive outer surface 269 of the device 260. Multiple holes or perforations 265 are defined through the flexible PCB 261 and any associated encapsulant material layers. One or more sensors 263 (e.g., temperature sensors or any other types of sensors disclosed herein) are arranged in or on the PCB 261. A socket 268 associated with the light emitting device 260 is arranged to receive a plug 277 to which the electrical cable 276 from the battery/control module 270 is attached. The battery/control module 270 includes a body 271, a battery 272, and a control board 273, which may include an emitter driver circuit and/or any suitable control, sensing, interface, data storage, and/or communication components as disclosed herein. The battery/control module 270 may further include a port or other interface 278 to enable communication with an external device (e.g., laptop or tablet computer, a mobile phone, or another portable digital device) via wired or wireless means.

FIG. 26B is a top schematic view of at least a portion of a light emitting device 280 for delivering light energy to living mammalian tissue and at least a portion of a battery/control module 290, wherein an elongated electrical cable 286 is associated with the light emitting device 280 for connecting the light emitting device 280 to the battery/control module 290. The light emitting device 280 is edge lit along one edge with a light emitting region 281A supported by a flexible PCB 281. The PCB 281 is preferably encapsulated on one or both sides with an encapsulant material. The flexible PCB 281 preferably includes a reflective material arranged to reflect light toward a light transmissive outer surface 289 of the device 280. Multiple holes or perforations 285 are defined through the flexible PCB 281 and any associated encapsulant material layers. One or more sensors 283 (e.g., temperature sensors or any other types of sensors disclosed herein) are arranged in or on the PCB 281. A socket 298 associated with the battery/control module 290 is arranged to receive a plug 287 to which the electrical cable 286 from the light emitting device 280 is attached. The battery/control module 290 includes a body 291, a battery 292, and a control board 293, which may include an emitter driver circuit and/or any suitable control, sensing, interface, data storage, and/or communication components as disclosed herein. The light emitting device 280 may further include a port or other interface 288 to enable communication with an external device (e.g., laptop or tablet computer, a mobile phone, or another portable digital device) via wired or wireless means.

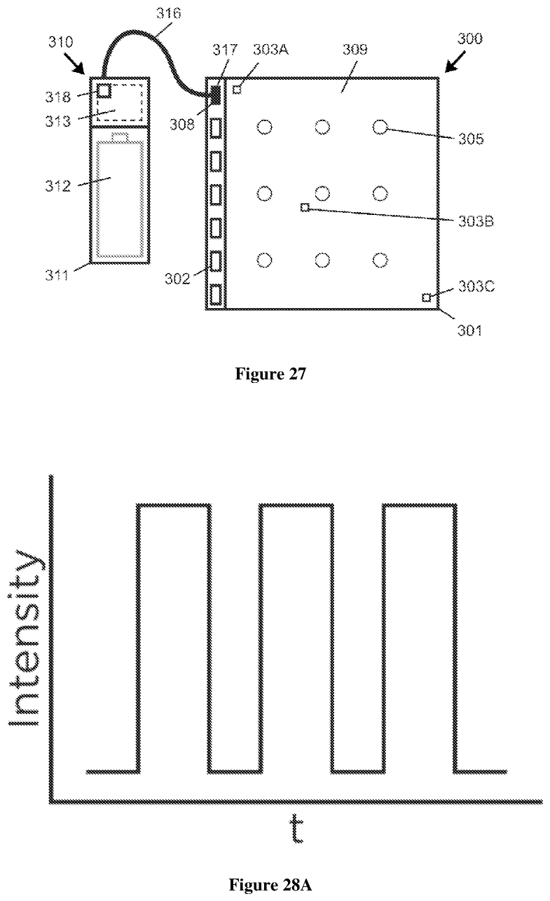

FIG. 27 is a top schematic view of at least a portion of a light emitting device 300 for delivering light energy to living mammalian tissue and being connected via an electrical cord 316 to a battery/control module 310, wherein the light emitting device 300 includes multiple light emitters 302 supported by a flexible PCB 301, multiple holes or perforations 305, and multiple sensors 303A-303C. The PCB 301 is preferably encapsulated on one or both sides with an encapsulant material. The flexible PCB 301 preferably includes a reflective material arranged to reflect light toward a light transmissive outer surface 309 of the device 300. Multiple holes or perforations 305 are defined through the flexible PCB 301 and any associated encapsulant material layers. Multiple sensors 303A-303C are arranged in or on the PCB 301. In certain embodiments, the sensors 303A-303C may differ in type from one another. In certain embodiments, the sensors 303A-303C may include one or more light emitters and photodiodes to illuminate a wound site with one or more selected wavelengths to detect blood flow in or proximate to a wound site to provide photoplethsmyography data. The sensors 303A-303C may alternatively or additionally be arranged to detect blood pressure, bandage or dressing covering pressure, heart rate, temperature, presence or concentration of chemical or biological species (e.g., in wound exudate), or other conditions. A socket 308 associated with the light emitting device 300 is arranged to receive a plug 317 to which the electrical cable 316 from the battery/control module 310 is attached. The battery/control module 310 includes a body 311, a battery 312, and a control board 313, which may include an emitter driver circuit and/or any suitable control, sensing, interface, data storage, and/or communication components as disclosed herein. The battery/control module 310 may further include a port or other interface 318 to enable communication with an external device (e.g., laptop or tablet computer, a mobile phone, or another portable digital device) via wired or wireless means.

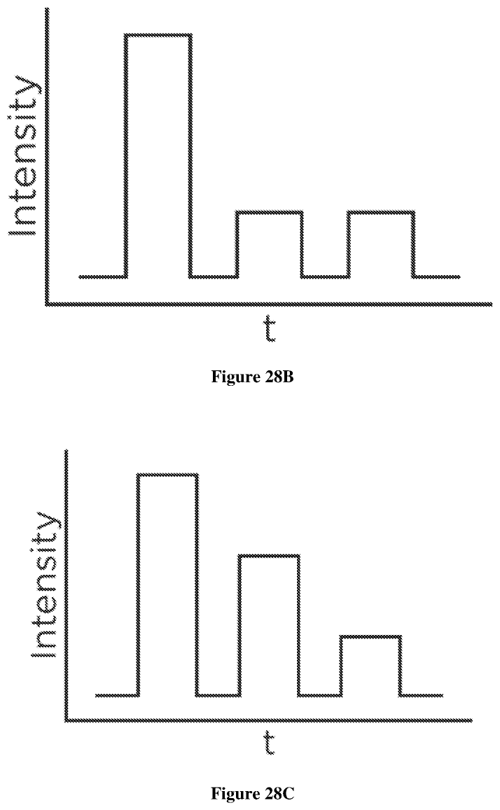

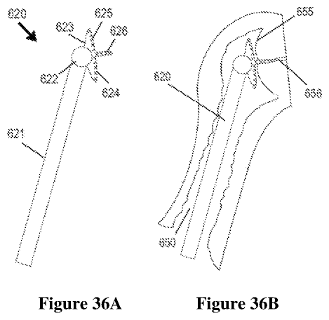

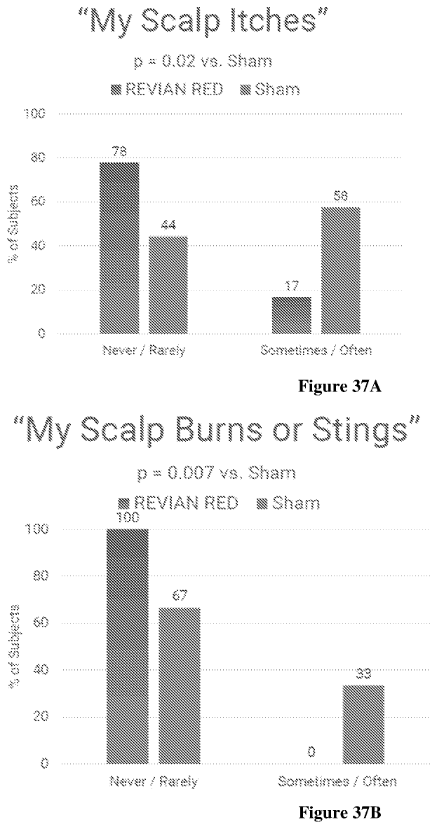

FIGS. 28A-28C illustrate different pulse profiles that may be used with devices and methods according to the present disclosure. FIG. 36A is a plot of intensity versus time embodying a first exemplary illumination cycle that may be used with at least one emitter of a light emitting device for delivering light energy to living mammalian tissue as disclosed herein. As shown in FIG. 28A, a series of discrete pulses of substantially equal intensity may be provided during at least one time window or a portion thereof. FIG. 28B is a plot of intensity versus time embodying a second exemplary illumination cycle that may be used with at least one emitter of a light emitting device disclosed herein. As shown in FIG. 28C, intensity may be reduced from a maximum (or high) value to a reduced but non-zero value during at least one time window. FIG. 28C is a plot of intensity versus time embodying a third exemplary illumination cycle that may be used with at least one emitter of a light emitting device disclosed herein. As shown in FIG. 28C, intensity may be steadily reduced from a maximum (or high) value to sequentially reduced values over time. Other pulse profiles may be used according to certain embodiments.

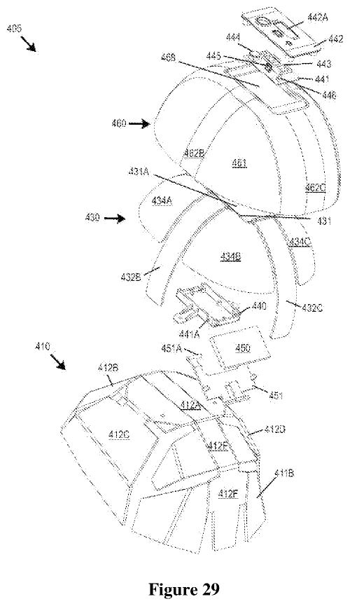

FIG. 29 is an exploded view of a light emitting device 405 embodied in a wearable cap for delivering light energy to a scalp of a patient. The device 405 includes multiple light emitters and standoffs supported by a flexible PCB 410 including multiple interconnected panels 412A-412F arranged in a concave configuration. A concave shaping member 430 (including a frame 431, ribs 432A-432D, and curved panels 434A-434D) is configured to receive the flexible PCB 410. The ribs 432A-432D and curved panels 434A-434D project generally outwardly and downwardly from the central frame 431. Gaps are provided between portions of adjacent ribs 432A-432D and curved panels 434A-434D to accommodate outward expansion and inward contraction, and to enable transfer of heat and/or fluid (e.g., evaporation of sweat). A fabric covering member 460 is configured to cover the concave shaping member 430 and the flexible PCB 410 contained therein. A battery 450 and a battery holder 451 are arranged between the flexible PCB 410 and the concave shaping member 430. An electronics housing 440 is arranged to be received within an opening 431A defined in a frame 431 of the concave shaping member 430. Pivotal coupling elements 441A, 451A are arranged to pivotally couple the battery holder 451 to the electronics housing 440. An electronics board 441 is insertable into the electronics housing 440, which is enclosed with a cover 442. Arranged on the electronics board 441 are a cycle counter 443, a control button 444, a charging/data port 445, and a status lamp 446. The various elements associated with the electronics housing 440 and the electronics board 441 may be referred to generally as a “control module.” Windows 442A defined in the cover 442 provide access to the cycle counter 443, the control button 444, the charging/data port 445, and the status lamp 446. The fabric covering element 460 includes a fabric body 461 and multiple internal pockets 462A-462D arranged to receive portions of the ribs 432A-432D. An opening 468 at the top of the fabric covering element 460 is arranged to receive the cover 442.

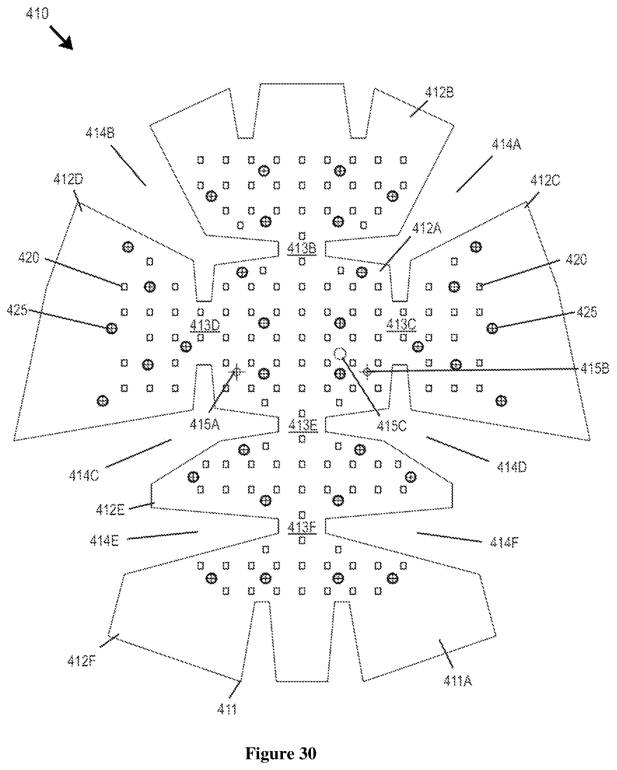

FIG. 30 is a bottom plan view of the flexible PCB 410 including light emitters 420 and standoffs 425 arranged thereon. The PCB 410 includes a polyimide substrate 411, an inner surface 411A, and an outer surface 411B. In one embodiment, the light emitters 420 include a total of 280 light emitting diodes arranged as 56 strings of 5 LEDs, with a string voltage of 11V, a current limit of 5 mA, and a power consumption of 3.08 watts. FIG. 38 illustrates 36 standoffs 425 extending from an inner surface 411A of the PCB 410. The flexible PCB 410 includes six interconnected panels 412A-412F, with the panels 412A-412F being connected to one another via narrowed tab regions 413B-413F. Gaps 414A-414F are provided between various panels 412A-412F, with such gaps 414A-414F (which are extended proximate to the narrowed tab regions 413B-413F) being useful to permit transport of heat and/or fluid (e.g., evaporation of sweat) between the panels 412A-412F. As shown in FIG. 38, holes 415A, 415B are defined through the substrate 411 to receive fasteners (not shown) for joining the PCB 410 to corresponding holes 440A, 440B defined in the electronics housing 440. A further opening 415C may be provided for sensor communication between a proximity sensor (e.g., photosensor) and the interior of the PCB 410 when the PCB 410 is shaped into in a concave configuration.

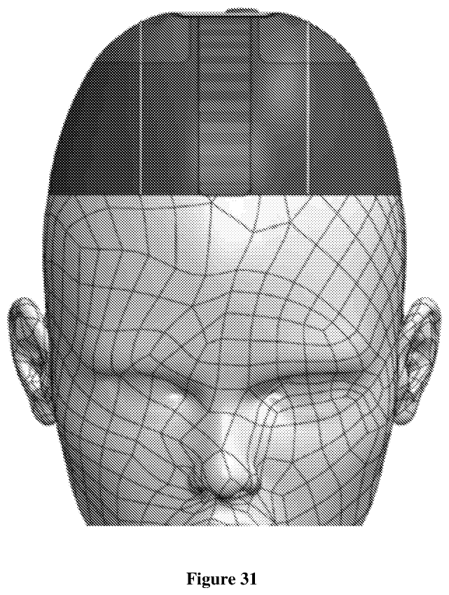

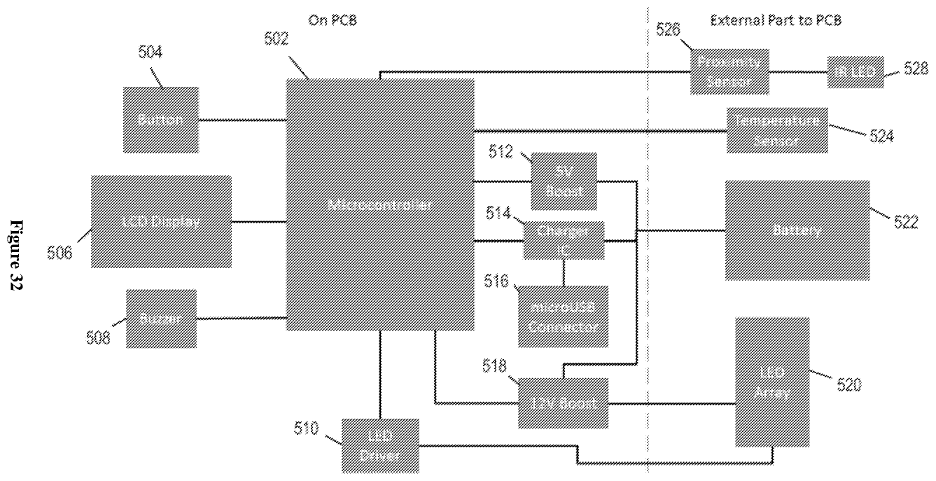

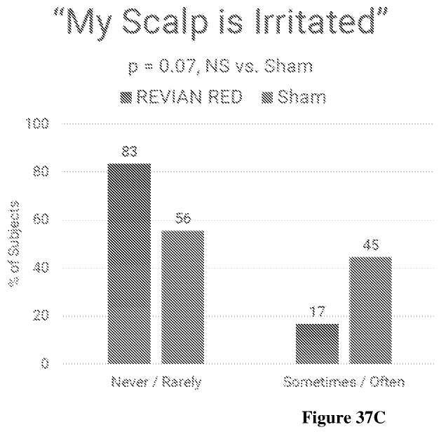

FIG. 31 is a front elevation view of the assembled light emitting device 405 embodied in a wearable cap of FIG. 37 superimposed over a modeled human head. As shown in FIG. 31, the device 405 is embodied in a cap with a lower edge between a user's forehead and hairline, and above a user's ears.

FIG. 32 is a schematic diagram showing interconnections between components of a light emitting device for delivering light energy to tissue of a patient according to one embodiment. A microcontroller 502 is arranged to receive power from a battery 522 (nominally 3.7V) via a 5V voltage boost circuit 522. The microcontroller may be arranged to control a charging integrated circuit 514 arranged between a microUSB connector 516 and the battery 522, wherein the microUSB connector 516 may be used to receive current for charging the battery. In certain embodiments, the microUSB connector 516 may also be used for communicating data and/or instructions to or from the microcontroller 502 and/or an associated memory. The microcontroller 502 is also arranged to control a 52V boost circuit 518 for increasing voltage to one or more LED arrays 520. The microcontroller 502 further controls one or more LED driver circuits 510 arranged to drive the LED array(s) 520. The microcontroller 502 is also arranged to receive inputs from a user input button 504, a temperature sensor 524, and a proximity sensor 526 (which includes an infrared LED 528). The microcontroller 502 is further arranged to provide output signals to a LCD display 506 and a buzzer 508. Certain components are located off-board relative to a controller PCB, as indicated by the vertical dashed line in FIG. 40. In operation of the light emitting device, a user may depress the button 504 to start operation. If the proximity sensor 526 detects that the device has been placed in suitable proximity to desired tissue, then the microcontroller may trigger the LED driver circuit(s) 510 to energize the LED array(s) 520. Temperature during operation is monitored with the temperature sensor 524. If an excess temperature condition is detected, then the microcontroller 502 may take appropriate action to reduce current supplied by the LED driver circuit(s) 510 to the LED array(s) 520. Operation may continue until a timer (e.g., internal to the microcontroller 502) causes operation to terminate automatically. One or more indicator LEDs (not shown) may provide a visible signal indicative of charging status of the battery 522. Audible signals for commencement and termination of operation may be provided by the buzzer 508 or a suitable speaker. Information relating to usage cycles, usage time, or any other suitable parameter may be displayed by the LCD display 506.

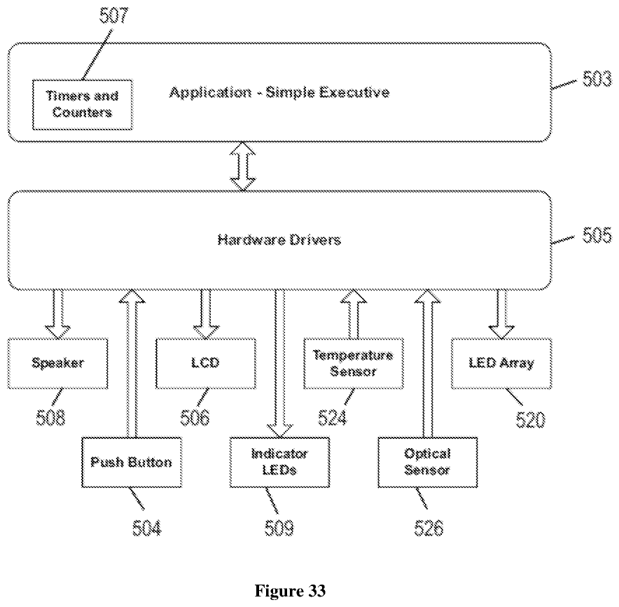

FIG. 33 is a schematic diagram depicting an interface between hardware drivers, functional components, and a software application suitable for operating a light emitting device according to FIG. 32. Application executive functions 503, including timers and counters 507, may be performed with one or more integrated circuits (such as the microcontroller 502 illustrated in FIG. 40). Hardware drivers 505 may be used to interface with various input and output elements, such as the LED array(s) 520, the speaker or buzzer 508, the LCD display 506, the temperature sensor 524, the push button 504, the indicator LEDs 509, and the optical sensor 526.

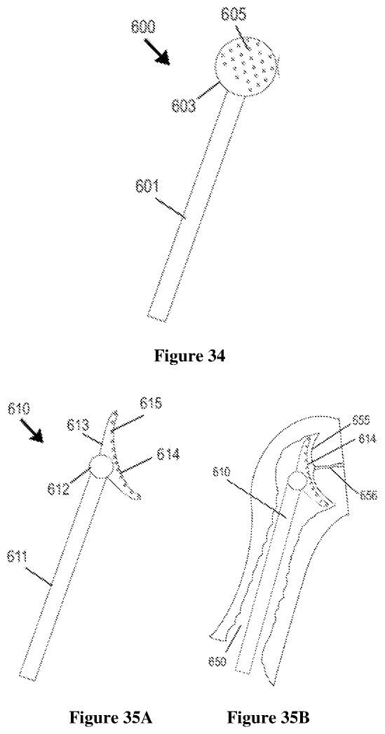

FIG. 34 is a schematic elevation view of at least a portion of a light emitting device 600 for delivering light energy to tissue in an internal cavity (e.g., body cavity) of a patient according to one embodiment. In certain embodiments, a body cavity may comprise a vaginal cavity, an oral cavity, or an esophageal cavity. If used in an oral or esophageal cavity, one or more unobstructed channels or tubes (not shown) may be provided in, on, or through the device 600 to avoid interruption with patient breathing. The device 600 includes a body 601 that may be rigid, semi-rigid, or articulated. A treatment head 603 has arranged therein or thereon one or more light emitters 605, which are preferably encapsulated in silicone or another suitable light transmissive material. In certain embodiments, the one or more light emitters 605 may be arranged to produce anti-inflammatory and ES increasing light or ES releasing light for impingement on tissue located within an internal cavity of a patient to trigger release of NO.

FIG. 35A is a schematic elevation view of at least a portion of a light emitting device 610 including a concave light emitting surface 614 including one or more light emitters 615 for delivering light energy to cervical tissue of a patient according to one embodiment. The device 610 includes a body 611 that may be rigid, semi-rigid, or articulated. A joint 612 may be arranged between the body 611 and a treatment head 613. The treatment head 613 has arranged therein or thereon one or more light emitters 615, which are preferably encapsulated in silicone or another suitable light transmissive material. In certain embodiments, the one or more light emitters 615 may be configured to generate emissions suitable for neutralizing pathogens such as human papilloma virus (HPV) present on cervical tissue. In certain embodiments, the one or more light emitters 615 may be arranged to produce ES increasing light and ES releasing light for impingement on tissue located within an internal cavity of a patient to trigger release of NO.

FIG. 35B illustrates the device of FIG. 35A inserted into a vaginal cavity 650 to deliver light energy to cervical tissue 655 of a patient proximate to a cervical opening 656. The concave light emitting surface 614 may be configured to approximately match a convex profile of the cervical tissue 655.

FIG. 36A is a schematic elevation view of at least a portion of a light emitting device 620 including a light emitting surface 624 with a protruding probe portion 626 for delivering light energy to cervical tissue of a patient according to another embodiment. The probe portion 626 includes light emitters and is arranged to deliver light energy into a cervical opening. The device 620 includes a body 621 that may be rigid, semi-rigid, or articulated. A joint 622 may be arranged between the body 621 and a treatment head 623. The treatment head 623 has arranged therein or thereon one or more light emitters 625, which are preferably encapsulated in silicone or another suitable light transmissive material. The treatment head 623 may include a primary light emitting surface 624, which may optionally be convex to cast a wider output beam. In certain embodiments, the one or more light emitters 625 may be configured to generate emissions suitable for neutralizing pathogens such as human papilloma virus (HPV) present on cervical tissue. In certain embodiments, the one or more light emitters 625 may be arranged to produce ES increasing light and ES releasing light for impingement on tissue located within an internal cavity of a patient to trigger release of NO.

FIG. 36B illustrates the device of FIG. 36A inserted into a vaginal cavity 650 to deliver light energy to cervical tissue 655 of a patient proximate and within to a cervical opening 656. The primary light emitting surface may be arranged to impinge light on cervical tissue bounding the vaginal cavity, whereas the probe portion may be inserted into the cervical opening to deliver additional light energy therein to increase the amount of cervical tissue subject to receipt of light energy for addressing one or more conditions including pathogen (e.g., HPV) neutralization.

The present invention will be better understood with reference to the following non-limiting examples.

### EXAMPLE 1

### Evaluation of Burning, Stinging and Pruritis Following Treatment

Scalp burning, stinging and pruritus are common patient complaints in the dermatological setting and can be frustrating for both the patient and the dermatologist. Indeed, the prevalence of pruritus of the scalp is up to 45% of patients with chronic pruritus.¹ These symptoms are often associated with conditions such as seborrheic dermatitis and scalp psoriasis, where up to 80% of patients with psoriasis report scalp itch with a positive correlation between the severity of the lesions and severity of itch², but these symptoms also may appear without any clinical findings. Treatment options for scalp disorders and associated symptoms include topical corticosteroids and, in some cases, anti-fungals, but the wide variety of underlying disease pathologies and limited compliance with dosing regimens hinder their clinical benefit.

Indeed, patients with androgenetic alopecia often complain of scalp itch and irritation and may also have concomitant seborrheic dermatitis. Based on this, the symptoms of pruritus, irritation, burning of the scalp were measured in an ongoing multicenter study evaluating the safety and efficacy of a dual wavelength LED light device in subjects being treated with androgenetic alopecia.

Light administered at dual wavelengths (for example, 620 nm and 660 nm) stimulates nitric oxide production and decreases inflammation.

Eighty-one subjects were randomized to either a dual wavelength 620 nm and 660 nm light therapy device paired with a Bluetooth-connected mobile app (REVIAN RED System) or to a sham comparator device with a similar user experience through the mobile app to track daily treatment compliance between both groups. Device usage was fixed at once daily, 10-minute treatment durations for a period of 26-weeks. The trial population consisted of adult men and women between 18 and 65 years of age with a diagnosis of androgenetic alopecia, consistent with males who have Norwood Hamilton Classification IIa to V patterns of hair loss and females who have Ludwig-Savin Scale I-1 to I-4, II-1, II-2 or frontal, both with Fitzpatrick Skin Types I-IV.

The Hair Specific Skindex-29 Quality of Life Questionnaire (HSSQOL) was used to assess Itching, Burning/Stinging, Irritation, and other patient reported outcomes. Participants scored each question on a scale from 1 (never) to 5 (all the time). The results are illustrated in FIGS. 37A-C, and discussed below.

Results:

Secondary Efficacy Assessment—Hair Specific Skindex-29 QOL At Week 16

The Hair Specific Skindex-29 Quality of Life Questionnaire (HSSQOL) is a 29-item questionnaire with 3 domains: 7 questions for symptoms domain, 10 questions for emotion domain and 12 questions for function domain. The Hair Specific Skindex 29 Quality of Life Questionnaire (HSSQOL) is a 29 item questionnaire with 3 domains: 7 questions for symptoms domain, 10 questions for emotion domain and 12 questions for function domain. Specifically, for the symptom of “my scalp burns or stings”, at the end of the 16-week trial 100% of the active treated group showed never or rarely having the symptom versus 66.6% of the sham group and 0% of the active group reported the symptoms sometime or often versus 33.4% of the sham treated group (p=0.007). For the symptom of “my scalp itches” (pruritus), 77.8% of the active treatment group and 44% of the sham treated group reported the symptom never or rarely versus 16.7% of the active group and 57.6% of the sham treaty group reporting the symptom sometime or often (p=0.02) Finally regarding my scalp is irritated 83.4% of the active treatment group and 55.5% of the sham treated group reported the symptom never or rarely versus 16.6% of the active treated group and 44.5% of the sham treat a group reported the symptoms sometime or often (P=0.07).

Red and Infrared Low Level Light Therapy (LLLT) previously been shown to have anti-inflammatory effects in patients with plaque psoriasis, leading to clearance of recalcitrant lesions³ and reductions in plaque desquamation, induration, and erythema.⁴ The addition of 620 nm LED light results in increased release of nitric oxide (NO) in the skin and provides a complimentary mechanism to reduce inflammation, irritation and pruritus.

The immunomodulatory modes of action associated with nitric oxide⁵ include decreased production of IL-1β, decreased production of IL-17, decreased E-selection expression of endothelial cells, and regulation of matrix metalloproteinase activity.

Light at 620 nm increases either the production and release of nitric oxide, and increases blood flow. Light at 660 nm increases ATP levels, increases cellular respiration, and decreases inflammatory cytokines.

### Conclusions

The FDA-cleared dual wavelength device (K173729) was found to be safe and well tolerated, with statistically significant differences observed in patient reported pruritus and burning/stinging compared to sham after 16 weeks of once daily, at home treatment.

The MOA for improved scalp symptoms are proposed to be a combination of the benefits of traditional anti-inflammatory and antipruritic effects of red (660 nm) LLLT and the anti-inflammatory effects of nitric oxide (NO) released with 620 nm light.

Applicants are unaware of any previous reports of a reduction in scalp pruritus with traditional LLLT devices used to treat androgenetic alopecia. The methods and devices described herein can be used to treat individuals suffering from itch and irritation symptoms associated with scalp conditions, such as seborrheic dermatitis or psoriasis.

### REFERENCES

1. Matterne et al. (2011) Prevalence, correlates and characteristics of chronic pruritus: A population based cross-sectional study. Acta Dermato-Venereologica 91: 674-679.
2. Kim et al. (2014) Clinical characteristics of pruritus in patients with scalp psoriasis and their relation with intraepidermal nerve fiber density. Ann Dermatol 26: 727-732
3. Ablon G. (2010) Combination 830 nm and 633 nm light-emitting diode phototherapy shows promise in the treatment of recalcitrant psoriasis: preliminary findings. Photomed Laser Surg 28:141-146
4. Kleinpenning et al. (2012) Efficacy of blue light vs. red light in the treatment of psoriasis: a double-blind, randomized comparative study. J Eur Acad Dermatol Venereol 26: 219-225
5. Del Rosso J Q, Kircik L (2017) Spotlight on the Use of Nitric Oxide in Dermatology: What Is It? What Does It Do? Can It Become an Important Addition to the Therapeutic Armamentarium for Skin Disease? J Drugs Dermatol 16 (1 Suppl 1):s4-10.

### Example 2: Comparative Results in a Pruritis Study

A further comparative study was performed using a “sham” cap (Cap 100), a cap with two wavelengths (620 and 660 nm; “Cap 101”), a cap with light at a blue wavelength, and a cap with a mixture of blue light and the two wavelengths 620 and 660 nm.

As shown in the following table, the results using a combination of 620 nm and 660 nm were much better than when blue light was used (“Cap 102”) and a mixture of all three wavelengths was used (“Cap 103”).

SECONDARY EFFICACY - Hair Specific Skindex [symbol] 29 [symbol] At Week 16
PP Population

| | All | | REVIAN | | REVIAN | | REVIAN | REVIAN |
|---|---|---|---|---|---|---|---|---|
| Statement | Active Caps | | Cap 101 | | Cap 102 | | Cap 103 | Sham Cap 190 |
| Scores | (N [symbol] 51) | | (N [symbol] 1 [symbol] ) | | (N [symbol] 1 [symbol] ) | | (N [symbol] 17) | (N [symbol] 1 [symbol] ) |
| [symbol] : My scalp itches. | | | | | | | | |
| Never (1) | 19 | (37.3%) | 9 | ( [symbol] 0.0%) | 6 | (37.5%) | 4 (23.5%) | 4 (22.2%) |
| Rarely (2) | 11 | (21.6%) | 5 | (27.8%) | 2 | (12.5%) | 4 (23.5%) | 4 (22.2%) |
| Sometimes (3) | 16 | (31.4%) | 3 | ( [symbol] .7%) | 7 | (43. [symbol] %) | 6 (35.3%) | 5 (27.8%) |

| | | | | | | | |
|---|---|---|---|---|---|---|---|
| Often (4) | 4 | (7.8%) | 0 | 1 | ( [symbol] . [symbol] %) | 5 (17.6%) | 5 (27.8%) |

| | | | | | | | |
|---|---|---|---|---|---|---|---|
| All the Time (5) | 1 | (2.0%) | 1 | ( [symbol] . [symbol] %) | 0 | [symbol] | 0 |

| | | | | |
|---|---|---|---|---|
| P-value vs Sham Cap (4) | 0.128 | 0.0 [symbol] 4 | 0.265 | 0.6 [symbol] 4 |

[symbol]

indicates data missing or illegible when filed

Those skilled in the art will recognize improvements and modifications to the preferred embodiments of the present disclosure. All such improvements and modifications are considered within the scope of the concepts disclosed herein and the claims that follow.

1. A method of treating skin disorders, the method comprising:
impinging light having a first peak wavelength on the tissue at a first radiant flux, wherein the first peak wavelength and the first radiant flux is selected to provide an anti-inflammatory effect, and
impinging light having a second peak wavelength on the tissue at a second radiant flux, wherein the second peak wavelength and the second radiant flux are selected to either stimulate enzymatic generation of nitric oxide to increase endogenous stores of nitric oxide or release nitric oxide from the endogenous stores,
wherein the skin disorders are selected from the group consisting of pruritus, psoriasis, acne, rosacea, eczema, such as eczema verruca vulgaris, neurofibromatosis, pyogenic granulomas, recessive dystrophic epidermolysis bullosa, venous ulcers, molluscum contagiosum, seborrheic keratosis, Sturge-Weber syndrome, actinic keratosis, and dandruff.

2. The method of claim 1, wherein the treatment reduces stinging and/or itching associated with the skin disorder.

3. The method of claim 1, wherein the skin disorders are selected from the group consisting of pruritus, psoriasis, acne, rosacea, and eczema.

4. The method of claim 1, wherein the skin disorders are disorders related to skin of the scalp.

5. The method of claim 1, wherein the light at the first wavelength and the light at the second wavelength are administered in combination or alternation.

6. The method of claim 1, wherein the light at the first wavelength is in the range of between about 650 and about 680 nm.

7. The method of claim 1, wherein the light at the first wavelength is in the range of between about 655 and about 665 nm.

8. The method of claim 1, wherein the light at the second wavelength is in the range of between about 615 and about 630 nm.

9. The method of claim 1, wherein the light at the second wavelength is about 620 nm.

10. The method of claim 1, wherein each of the first radiant flux and the second radiant flux is in a range of from 5 mW/cm² to 60 mW/cm².

11. The method of claim 1, wherein the light having a first peak wavelength is produced by a first array of light emitting devices, and the light having a second peak wavelength is produced by a second array of light emitting devices, wherein the light having a first peak wavelength comprises a first spectral output having a first full width at half maximum value of less than 25 nm, and the light having a second peak wavelength comprises a second spectral output having a second full width at half maximum value of less than 25 nm.

12. The method of claim 11, wherein less than 5% of the first spectral output is in a wavelength range of less than 400 nm, and less than 1% of the second spectral output is in a wavelength range of less than 400 nm.

13. The method of claim 1, wherein the second peak wavelength is in a range of from 400 nm to 420 nm or from 510 nm to 550 nm.

14. The method of claim 1, wherein the tissue comprises epithelial tissue or tissue of the scalp.

15. A device for modulating nitric oxide in living mammalian tissue, the device comprising:
means for impinging light having a first peak wavelength on the tissue at a first radiant flux, wherein the first peak wavelength and the first radiant flux are selected to provide anti-inflammatory effects, and
means for impinging light having a second peak wavelength on the tissue at a second radiant flux, wherein the second peak wavelength and the second radiant flux are selected to either stimulate enzymatic generation of nitric oxide to increase endogenous stores of nitric oxide or release nitric oxide from the endogenous stores.

16. The device of claim 15, wherein each of the first radiant flux and the second radiant flux is in a range of from 5 mW/cm² to 60 mW/cm².

17. The device of claim 16, wherein the light having a first peak wavelength comprises a first spectral output having a first full width at half maximum value of less than 25 nm, and the light having a second peak wavelength comprises a second spectral output having a second full width at half maximum value of less than 25 nm.

18. The device of claim 17, wherein the first peak wavelength is in a range of from 400 nm to 490 nm, and the second peak wavelength is in a range of from 500 nm to 900 nm.

19. The device of claim 15, further comprising means for sensing a temperature condition on or proximate to (a) the device or (b) the tissue; means for generating at least one signal indicative of the temperature condition; and means for controlling at least one of the following items (i) and (ii) responsive to the at least one signal: (i) impingement of light having the first peak wavelength on the tissue, and (ii) impingement of light having the second peak wavelength on the tissue.

20. A device for treating skin disorders in living mammalian tissue, the device comprising:
at least one first light emitting device configured to impinge light having a first peak wavelength on the tissue at a first radiant flux, wherein the first peak wavelength and the first radiant flux are selected to provide anti-inflammatory effects; and
at least one second light emitting device configured to impinge light having a second peak wavelength on the tissue at a second radiant flux, wherein the second peak wavelength and the second flux are selected to either stimulate enzymatic generation of nitric oxide to increase endogenous stores of nitric oxide or release nitric oxide from the endogenous stores.

21. The device of claim 20, further comprising driver circuitry configured to drive the at least one first light emitting device and the at least one second light emitting device.

22. The device of claim 20, wherein each of the first radiant flux and the second radiant flux is in a range of from 5 mW/cm² to 60 mW/cm².

23. The device of claim 20, wherein the second peak wavelength exceeds the first peak wavelength by at least 50 nm.

24. The device of claim 23, wherein the light having a first peak wavelength comprises a first spectral output having a first full width at half maximum value of less than 25 nm, and the light having a second peak wavelength comprises a second spectral output having a second full width at half maximum value of less than 25 nm.
